SEQUENCE LISTING (16 sequences)

SEQ ID NO: 1 - DNA, 25 bp, synthetic construct
  misc_feature 1..25: note = Synthetic: MATa F primer
  source 1..25: mol_type = other DNA
ctccacttcaagtaagagtttgggt

SEQ ID NO: 2 - DNA, 24 bp, synthetic construct
  misc_feature 1..24: note = Synthetic: MATalpha F primer
  source 1..24: mol_type = other DNA
ttactcacagtttggctccggtgt

SEQ ID NO: 3 - DNA, 24 bp, synthetic construct
  misc_feature 1..24: note = Synthetic: Common MAT R primer
  source 1..24: mol_type = other DNA
gaaccgcatgggcagtttaccttt

SEQ ID NO: 4 - DNA, 27 bp, synthetic construct
  misc_feature 1..27: note = Synthetic: oHDP022 primer
  source 1..27: mol_type = other DNA
tgattatagccacgggtgaratgttyt

SEQ ID NO: 5 - DNA, 27 bp, synthetic construct
  misc_feature 1..27: note = Synthetic: oHDP023 primer
  source 1..27: mol_type = other DNA
tgattatagccackggtgaratgtttt

SEQ ID NO: 6 - DNA, 27 bp, synthetic construct
  misc_feature 1..27: note = Synthetic: oHDP024 primer
  source 1..27: mol_type = other DNA
ttcaktcatcaaytttgaggcccatgt

SEQ ID NO: 7 - DNA, 20 bp, synthetic construct
  misc_feature 1..20: note = Synthetic: oHWA230 primer
  source 1..20: mol_type = other DNA
aaacgctcccctcacagacg

SEQ ID NO: 8 - DNA, 20 bp, synthetic construct
  misc_feature 1..20: note = Synthetic: oHWA231 primer
  source 1..20: mol_type = other DNA
ctgggcagatgatgtcgagg

SEQ ID NO: 9 - DNA, 60 bp, synthetic construct
  misc_feature 1..60: note = Synthetic: CTH993 primer
  source 1..60: mol_type = other DNA
caaatacacacactaaattaccggatcaattcgggggaaaaatgctttctgaaaacacga

SEQ ID NO: 10 - DNA, 51 bp, synthetic construct
  misc_feature 1..51: note = Synthetic: CTH994 primer
  source 1..51: mol_type = other DNA
cctccacctccaccgttaattaacccggggatccggcagatgcgcgcacct

SEQ ID NO: 11 - protein, 586 aa, Saccharomyces cerevisiae
  REGION 1..586: note = misc_feature - Saccharomyces cerevisiae HO protein
MLSENTTILMANGEIKDIANVTANSYVMCADGSAARVINVTQGYQKIYNIQQKTKHRAFEGEPGRLDPRRRTVYQRLALQCTAGHKLSVRVPTKPLLEKSGRNATKYKVRWRNLQQCQTLDGRIIIIPKNHHKTFPMTVEGEFAAKRFIEEMERSKGEYFNFDIEVRDLDYLDAQLRISSCIRFGPVLAGNGVLSKFLTGRSDLVTPAVKSMAWMLGLWLGDSTTKEPEISVDSLDPKLMESLRENAKIWGLYLTVCDDHVPLRAKHVRLHYGDGPDENRKTRNLRKNNPFWKAVTILKFKRDLDGEKQIPEFMYGEHIEVREAFLAGLIDSDGYVVKKGEGPESYKIAIQTVYSSIMDGIVHISRSLGMSATVTTRSAREEIIEGRKVQCQFTYDCNVAGGTTSQNVLSYCRSGHKTREVPPIIKREPVYFSFTDDFQGESTVYGLTIEGHKNFLLGNKIEVKSCRGCCVGEQLKISQKKNLKHCVACPRKGIKYFYKDWSGKNRVCARCYGRYKFSGHHCINCKYVPEAREVKKAKDKGEKLGITPEGLPVKGPECIKCGGILQFDAVRGPHKSCGNNAGARIC

SEQ ID NO: 12 - DNA, 1758 bp, Saccharomyces cerevisiae
  misc_feature 1..1758: note = Saccharomyces cerevisiae HO polynucleotide sequence
  source 1..1758: mol_type = genomic DNA
atgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcgcaggaaatggtgttttatctaaatttctcactggacgtagtgaccttgtaactcctgctgtaaaaagtatggcttggatgcttggtctgtggttaggtgacagtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaacttcacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcttaaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgc

SEQ ID NO: 13 - DNA, 13 bp, Saccharomyces cerevisiae
  misc_feature 1..13: note = S. cerevisiae Ho recognition site in MATa
  source 1..13: mol_type = genomic DNA
tttccgcaacagt

SEQ ID NO: 14 - DNA, 13 bp, Saccharomyces cerevisiae
  misc_feature 1..13: note = S. cerevisiae Ho recognition site in MATa
  source 1..13: mol_type = genomic DNA
ttcgcgcaacagt

SEQ ID NO: 15 - DNA, 11349 bp, synthetic construct
  misc_feature 1..11349: note = Synthetic: pHRW40(Kan), artificial plasmid sequence
  source 1..11349: mol_type = other DNA
gaattcttattacgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggcgatcaaataattcgatagcttgtcgtaataatggcggcatactatcagtagtaggtgtttccctttcttctttagcgacttgatgctcttgttcttccaatacgcaacctaaagtaaagtgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatgggtaaggcgtcgagcaaagcccgcttattttttacatgccaatacaatgtaggctgctctacacctagcttctgggcgagtttacgggttgttaaaccttcgattccgaccccattaagcagctctaatgcgccgttaatcactttacttttatctaatctagacatatgaattaattcgggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttagcgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcctcgagtaattcgcgccacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcagatcaattcctcgatcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtaccctatggcatgcatgtgctctgtatgtatataaaactcttgttttcttcttttctctaaatattctttccttatacattaggtcctttgtagcataaattactatacttctatagacacgcaaacacaaatacacacactaaattaccggatcaattcgggggaatgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcgcaggaaatggtgttttatctaaatttctcactggacgtagtgaccttgtaactcctgctgtaaaaagtatggcttggatgcttggtctgtggttaggtgacagtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaacttcacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcttaaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgccggatccccgggttaattaacggtggaggtggaggtggaggtggaggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatcaatcactagtgaattcgcgccacttctaaataagcgaatttcttgagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagctgaagcttcgtacgctgcaggtcgacggatccccgggttaattaaggcgcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattgaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaattcatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgatatcagatccactagtggcctatgcaattctcatgtttgacagcttatcatcgatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaagccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattatcgatgccggtgttattcttgctatgataggccatcgaatgttaaagggaagatttattgttttgccctttaaagacgaatttggaccgtgaatgtttaaataattctcatgtttgacagcttatcatcgaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccagcgatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 16 - DNA, 10914 bp, synthetic construct
  misc_feature 1..10914: note = Synthetic: pHRW32(Zeo), Artificial plasmid sequence
  source 1..10914: mol_type = other DNA
gaattcttattacgatcctcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggcgatcaaataattcgatagcttgtcgtaataatggcggcatactatcagtagtaggtgtttccctttcttctttagcgacttgatgctcttgttcttccaatacgcaacctaaagtaaagtgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatgggtaaggcgtcgagcaaagcccgcttattttttacatgccaatacaatgtaggctgctctacacctagcttctgggcgagtttacgggttgttaaaccttcgattccgaccccattaagcagctctaatgcgccgttaatcactttacttttatctaatctagacatatgaattaattcgggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttagcgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcctcgagtaattcgcgccacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcagatcaattcctcgatcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtaccctatggcatgcatgtgctctgtatgtatataaaactcttgttttcttcttttctctaaatattctttccttatacattaggtcctttgtagcataaattactatacttctatagacacgcaaacacaaatacacacactaaattaccggatcaattcgggggaatgctttctgaaaacacgactattctgatggctaacggtgaaattaaagacatcgcaaacgtcacggctaactcttacgttatgtgcgcagatggctccgctgcccgcgtcataaatgtcacacagggctatcagaaaatctataatatacagcaaaaaaccaaacacagagcttttgaaggtgaacctggtaggttagatcccaggcgtagaacagtttatcagcgtcttgcattacaatgtactgcaggtcataaattgtcagtcagggtccctaccaaaccactgttggaaaaaagtggtagaaatgccaccaaatataaagtgagatggagaaatctgcagcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcgcaggaaatggtgttttatctaaatttctcactggacgtagtgaccttgtaactcctgctgtaaaaagtatggcttggatgcttggtctgtggttaggtgacagtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaagatctcttggtatgtcagctactgtgacgaccaggtcagctagggaggaaatcattgaaggaagaaaagtccaatgtcaatttacatacgactgtaatgttgctgggggaacaacttcacagaatgttttgtcatattgtcgaagtggtcacaaaacaagagaagttccgccaattataaaaagggaacccgtatatttcagcttcacggatgatttccagggtgagagtactgtatatgggcttacgatagaaggccataaaaatttcttgcttggcaacaaaatagaagtgaaatcatgtcgaggctgctgtgtgggagaacagcttaaaatatcacaaaaaaagaatctaaaacactgtgttgcttgtcccagaaagggaatcaagtatttttataaagattggagtggtaaaaatcgagtatgtgctagatgctatggaagatacaaattcagcggtcatcactgtataaattgcaagtatgtaccagaagcacgtgaagtgaaaaaggcaaaagacaaaggcgaaaaattgggcattacgcccgaaggtttgccagttaaaggaccagagtgtataaaatgtggcggaatcttacagtttgatgctgtccgcgggcctcataagagttgtggtaacaacgcaggtgcgcgcatctgccggatccccgggttaattaacggtggaggtggaggtggaggtggaggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatctcagaagaagacttgaacggactcgacggtgaacaaaagttgatttctgaagaagatttgaacggtgaacaaaagctaatctccgaggaagacttgaacggtgaacaaaaattaatcaatcactagtgaattcgcgccacttctaaataagcgaatttcttgagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagctgaagcttcgtacgctgcaggtcgacggatccccgggttaattaaggcgcgccagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggctaagttgacctctgctgttccagttttgactgctagagatgttgctggtgctgttgaattttggaccgacagattgggtttttccagagatttcgttgaagacgatttcgctggtgttgttcgtgacgatgttaccttgttcatctccgccgttcaagatcaagttgttccagataatactttggcttgggtttgggttagaggtttagacgagttgtatgctgaatggtctgaagtcgtttctactaacttcagagatgcttctggtccagctatgaccgaaattggtgaacaaccatggggtagagaatttgccttgagagacccagccggtaattgtgttcatttcgtcgccgaagaacaagattgatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgatatcagatccactagtggcctatgcaattctcatgtttgacagcttatcatcgatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaagccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattatcgatgccggtgttattcttgctatgataggccatcgaatgttaaagggaagatttattgttttgccctttaaagacgaatttggaccgtgaatgtttaaataattctcatgtttgacagcttatcatcgaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccagcgatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

(19) United States
(12) Patent Application Publication
(10) Pub. No.: US 2024/0200103 A1
(43) Pub. Date: Jun. 20, 2024

(54) SYNTHETIC YEAST CELLS AND METHODS OF MAKING AND USING THE SAME

(71) Applicant: Wisconsin Alumni Research Foundation, Madison, WI (US)

(72) Inventors: William Gerald ALEXANDER, Madison, WI (US); David Peris NAVARRO, Madison, WI (US); Christopher Todd HITTINGER, Madison, WI (US)

(21) Appl. No.: 18/417,305

(22) Filed: Jan. 19, 2024

Related U.S. Application Data
(63) Continuation of application No. 15/805,950, filed on Nov. 7, 2017, now Pat. No. 11,913,016.
(60) Provisional application No. 62/418,444, filed on Nov. 7, 2016.
(60) Provisional application No. 62/524,700, filed on Jun. 26, 2017.

(51) Int. Cl. C12N 15/90; C12N 1/18; C12N 9/16; C12N 15/65; C12N 15/81

(52) U.S. Cl. CPC C12N 15/905; C12N 1/18; C12N 9/16; C12N 15/65; C12N 15/81; C12N 2800/80; C12N 2840/002

(57) ABSTRACT

Methods of making synthetic yeast cells by mating together two diploid (or higher ploidy) yeast species or hybrids to generate multiploid yeast hybrids are provided herein. The synthetic yeast cells made by this process and kits for performing the process are also provided.

### CROSS-REFERENCE TO RELATED APPLICATIONS

This patent application is a continuation of U.S. patent application Ser. No. 15/805,950, filed Nov. 7, 2017, which claims the benefit of priority of U.S. Provisional Patent Application No. 62/418,444, filed Nov. 7, 2016, and U.S. Provisional Patent Application No. 62/524,700, filed Jun. 26, 2017, all of which are incorporated herein by reference in their entireties.

### STATEMENT REGARDING FEDERALLY SPONSORED RESEARCH

This invention was made with United States government support under grant number DE-FC02-07ER64494 awarded by the US Department of Energy and 1253634 awarded by the National Science Foundation. The government has certain rights in this invention.

### SEQUENCE LISTING

This application is being filed electronically via Patent Center and includes an electronically submitted Sequence Listing in .xml format. The .xml file contains a sequence listing entitled “960296.04489.xml” created on Jan. 17, 2024 and is 46,037 bytes in size. The Sequence Listing contained in this .xml file is part of the specification and is hereby incorporated by reference herein in its entirety.

### INTRODUCTION

Yeasts are critical biocatalysts commonly used in many types of commercial fermentations. One potential way of developing new yeast biocatalysts takes advantage of the natural mechanism of reproduction exhibited by many yeast species. For example, numerous yeast species are able to reproduce sexually, wherein meiosis serves to both increase genetic diversity and to repair genetic material. As with other eukaryotes, yeast meiosis generally initiates in diploid cells (John, 1990). Regulation of this process involves specialized genetic loci called the mating-type (MAT) loci. Among yeast species, Saccharomyces yeast species, for example, possess two different mating type loci, MATa and MATα which each may contain two different genes. These mating-type genes encode transcription factors that regulate the expression of meiosis-specific genes, as well as other genes that function in self-recognition, pheromone production, and non-homologous end joining.

In addition to performing sexual reproduction among members of the same species (“intraspecies sexual reproduction”), Saccharomyces yeasts may also perform sexual reproduction among members of different species (“interspecies sexual reproduction”). All seven species of Saccharomyces yeasts possess the same mating-type locus organization and a predominantly diploid or diplontic lifestyle. Only limited pre-zygotic speciation barriers exist between Saccharomyces species, making hybridization possible between different Saccharomyces yeast species (“interspecies hybrids”).

Interestingly, interspecies yeast hybrids produce many commercially important fermentation products. For example, Saccharomyces cerevisiae x Saccharomyces eubayanus hybrids are used to produce lager beer, the most common fermented beverage on the planet (Corran, 1975; Libkind et al., 2011). Two major lineages are used in lager production (Dunn and Sherlock, 2008; Nakao et al., 2009; Walther et al., 2014), and recent evidence indicates that these two lineages arose from independent hybridization events, suggesting that genetic diversity from the parental populations may be one contributor to the phenotypic differences seen in modern industrial strains (Baker et al., 2015). Many other fermented beverages also make use of Saccharomyces hybrids: S. cerevisiae x Saccharomyces uvarum hybrids are used in production of some ciders and cold-fermented wines (Le Jeune et al., 2007; Masneuf et al., 1998; Pérez-Través et al., 2014b), while many Belgian ale and some European wine yeasts are S. cerevisiae x Saccharomyces kudriavzevii hybrids (Peris et al., 2012a, 2012b).

Given the importance of interspecies yeast hybrids in many commercial fermentations, there is an interest in the art of developing new synthetic interspecies hybrids that may possess novel properties and allow for strain improvement. However, current methods for making synthetic interspecies hybrids are cumbersome and/or require genomic modifications. New hybrid brewing strains have been generated by a laborious process of isolating auxotrophic mutants, which arise spontaneously at low frequency, followed by sporulation, dissection, and crossing MAT-compatible spores to obtain hybrids (Krogerus et al., 2015; Pérez-Través et al., 2012). Although these methods lack markers, which would likely streamline approval for food and beverage applications, the strains contain mutations in important biosynthetic pathways. Furthermore, using genetic markers can generate a reduction in genetic diversity by co-selecting for physically linked chromosomal sequences that could inadvertently remove interesting industrial traits. An easier method is to first generate stable heterothallic haploids for one or both parents, such as by replacing HO with drug markers, followed by interspecies crosses (Bullard et al., 2010; Swain Lenz et al., 2014; Tirosh et al., 2009). Variations of this strategy have used complementary drug markers and auxotrophic mutants in one species and spore dissection of wild-type diploids from another (Hebly et al., 2015; Piatkowska et al., 2013). However, the persistence of drug markers in the latter hybrids raises legitimate concerns about their safety that would need to be addressed prior to introducing them into the food and beverage industry.

Accordingly, there is a need in the art for new, easier, and “scarless” methods of creating genetic diversity within a yeast cell as well as new synthetic yeast strains that may be used for commercial fermentations such as in the beverage and biofuel industries.

### SUMMARY

In one aspect, methods of making synthetic yeast cells are provided. The methods may be directed to mating together two diploid (or higher ploidy) yeast species or hybrids. The methods may include i) introducing a first polynucleotide comprising a first promoter operably connected to an HO polynucleotide encoding a yeast Ho protein, a first selectable marker cassette, and a first yeast origin of replication into a first yeast cell from a first yeast species or hybrid to produce a first transformed yeast cell, ii) introducing a second polynucleotide comprising a second promoter operably connected to an HO polynucleotide encoding a yeast Ho protein, a second selectable marker cassette, and a second yeast origin of replication into a second yeast cell from a second yeast species or hybrid to produce a second transformed yeast cell, and iii) co-culturing the first transformed yeast cell with the second transformed yeast cell to produce a mating mixture.

Alternatively, in another aspect, the methods of the present invention may also be directed to mating a diploid (or higher ploidy) yeast species with a haploid yeast species. Such methods may include i) introducing a first polynucleotide comprising a first promoter operably connected to an HO polynucleotide encoding a yeast Ho protein, a first selectable marker cassette, and a first yeast origin of replication into a first yeast cell from a first yeast species or hybrid to produce a first transformed yeast cell, ii) introducing a second polynucleotide comprising a second selectable marker cassette, and a second yeast origin of replication into a second yeast cell from a second haploid yeast species to produce a second transformed yeast cell, and iii) co-culturing the first transformed yeast cell with the second transformed yeast cell to produce a mating mixture.

In a further aspect, synthetic yeast cells are provided. The synthetic yeast cells may include 5, 6, 7, 8, 9, 10, 11, 12, 13, 14, 15, 16, or more sets of yeast chromosomes. As used herein, a “set of chromosomes” refers to a complete or substantially complete set of chromosomes. A set of chromosomes is 1N, a yeast is generally 2N (diploid), 4N (tetraploid), etc. The sets of chromosomes may all come from the same yeast species or may come from 2, 3, 4, 5, 6, 7, 8, or more different yeast species. The synthetic yeast cells of the present invention may also include chromosomes or chromosomal segments from 3, 4, 5, 6, 7, 8, or more yeast species.

In a further aspect, methods of using the synthetic yeast cells described herein are provided. The methods of use may include using any one of synthetic yeast cells described herein or any one of the synthetic yeast cells made by the methods disclosed herein in a fermentation process. In some embodiments, the fermentation process may be involved in beer making, wine making, cider making, biofuel production, biochemical production, or another commercially valuable process.

In a still further aspect, kits are provided. The kits may include a first polynucleotide comprising a first promoter operably connected to an HO polynucleotide encoding a yeast Ho protein, a first selectable marker cassette, and a first yeast origin of replication, and a second polynucleotide comprising a second promoter operably connected to an HO polynucleotide encoding a yeast Ho protein, a second selectable marker cassette, and a second yeast origin of replication. In some embodiments, the first selectable marker cassette and the second selectable marker cassette encode different selectable markers.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIGS. 1A-1D shows DNA content of cells from double-drug-resistant colonies arising from HO-induced and co-cultured diploids. Diploid parents are also displayed in FIGS. 1A-1D. Flow cytometry was used to measure the DNA content of fixed, SYBR Green-stained cells harvested during exponential growth. The approximately tetraploid Weihenstephan strain is included on all graphs as a standard (Walther et al., 2014) and serves as a control to demonstrate that the new synthetic hybrids are tetraploid. Note that strains NRRL YB-210 and Wyeast #1007 German Ale did not survive the standard fixation and staining procedure intact, but genome sequencing and tetrad dissection have shown that NRRL YB-210 is approximately diploid (Wohlbach et al., 2014). These strains were stained by removing heating and protease treatments, and their comparative ploidy was determined (FIG. 6). FIG. 1A shows yHWA358 and its diploid (2n) Ethanol Red parent, FIG. 1B shows yHWA375 and its diploid S. kudriavzevii parent, FIG. 1C shows yHWA377 and its diploid S. uvarum parent, and FIG. 1D shows yHWA425 and its diploid S. eubayanus parent. Scer=S. cerevisiae, Skud=S. kudriavzevii, Suva=S. uvarum, Seub=S. eubayanus.

FIGS. 2A-2B shows how PCR and Restriction Fragment Long Polymorphism analyses confirm the presence of two different genomes in the synthetic allotetraploids. FIG. 2A shows the BRE5 PCR product is approximately the same size in all species shown. FIG. 2B shows digestion of the BRE5 PCR product with HaeIII produces a unique pattern for each species. Double-drug-resistant allotetraploids produce banding patterns consistent with those expected from a hybrid strain. Scer=S. cerevisiae, Skud=S. kudriavzevii, Suva=S. uvarum, Seub=S. eubayanus.

FIG. 3 shows an overview of the Hybrid Production (HyPr) method. Induction of HO expression by a doxycycline-inducible promoter in two diploid cultures, followed by co-culture and subsequent double-drug selection, will produce hybrids at a rate approaching 1 out of 1000 cells plated. Plasmids can then be easily cured or spontaneously lost to produce strains without genome modifications.

FIGS. 4A-4C shows vector maps of pBM5155 (FIG. 4A), pHCT2 (FIG. 4B), and pHMK34 (FIG. 4C). Sequences for these vectors have been deposited in GenBank under the accession numbers KT725394, KT725395, and KT781077, respectively. Vector maps were drawn in Geneious 4.7.4.

FIG. 5 shows pHCT2 facilitates MAT locus switching in hoΔ haploid strains of Saccharomyces. 47 numbered colonies had genomic DNA extracted by yeast colony PCR, and their mating-type locus analyzed by PCR as described in Table 2; the Wyeast #1007 German Ale strain was included as a diploid control (G). MATa produced the larger PCR product (left band for each colony), while MATa produced the smaller PCR product (right band for each colony). Presence of a band for only one of the strains' PCR reactions indicates a haploid cell. Colonies 3, 7, 9, 15, 18, 24, 31, 39, 43, 44, 45, and 46 only contained MATa, indicating that mating-type switching and subsequent autodiploidization did not occur in these colonies.

FIG. 6 shows NRRL YB-210 and Wyeast #1007 are approximately diploids. The standard fixation and staining procedure led to cellular degradation of both NRRL YB-210 and Wyeast #1007. Removing the heating and Proteinase K treatments prevented this degradation. NRRL YB-210 is known to be approximately diploid based on genome sequencing and tetrad dissection (Wolhbach et al, 2014). Different ratios of cells in two cell cycle phases (G1 and G2) are apparent in the figure; NRRL YB-210 has more cells in G1 of the cell cycle, while Wyeast #1007 has more cells in G2 of the cell cycle.

FIG. 7 shows HyPr hybrids without plasmids can be easily isolated. A saturated yHWA425 culture was diluted 10⁻², and 10 μL of the dilution was plated to YPD (right). After three days of growth at room temperature, the plate was replicated to YPD +nat (left) and YPD +hyg (center). After two more days of growth, two colonies were picked on the basis of their drug sensitivity (circled colonies).

FIG. 8 shows how PCR and Restriction Fragment Long Polymorphism confirm the presence of three different genomes (S. cerevisiae x S. kudriavzevii x S. eubayanus) in a synthetic higher ploidy hybrid (likely an allohexaploid). The BRE5 PCR product was digested with HaeII producing a pattern for each species contributing to this gene/genome, as described in FIGS. 2A-2B.

FIG. 9 is a set of sequencing analyses showing that the four species hybrid contains DNA sequences associated with each of the four parent species (S. cerevisiae, S. paradoxus, S. arboricola and S. uvarum, respectively from top to bottom in the figure). Notably the selected hybrid had increased read coverage for one parent and decreased read coverage for at least one parent on some of the chromosomes.

FIG. 10 is a schematic depiction of the scheme for making the 6 species hybrid indicating the strains used in the hybrid production scheme and the resulting allododecaploid yeast.

FIG. 11A is a photograph of the 6 species hybrids showing the range of yeast morphology found in the allododecaploid yeast. FIG. 11B is a photograph of a Restriction Fragment Length Polymorphism (RFLP) analysis used to confirm the 6 species hybrid contained genetic material derived from the parental strains of yeast. This RFLP analysis confirms the presence of at least 5 of the parental lines.

FIG. 12 shows the plasmid maps for a second set of HyPr plasmids pHRW32 with Zeocin resistance, the sequence of which is provided as SEQ ID NO: 16 and pHRW40 with Kanamycin resistance, the sequence of which is provided as SEQ ID NO: 15.

### DETAILED DESCRIPTION

Here, the inventors describe new methods for the efficient production of designer yeast strains based on two expression plasmids. These plasmids may include complementary dominant drug-resistance cassettes, a promoter operably connected to an HO polynucleotide encoding a yeast Ho protein, and a generalized replication origin that provides functionality across Saccharomyces and many other yeasts.

In the non-limiting Examples, the inventors demonstrate that the methods disclosed herein efficiently produce allotetraploid and autotetraploid strains of Saccharomyces, as well as new Saccharomyces strains including more than 4 sets of chromosomes. The resulting strains can be rapidly screened for plasmid loss, providing an efficient route towards meeting the Generally Recognized As Safe (GRAS) standards of the United States Department of Agriculture and Food and Drug Administration. These methods and strains also provide a valuable and general research tool for basic and applied research on prototrophic hybrids and polyploids of yeasts.

The methods disclosed herein also provide an alternative method for optimization via hybridization of yeast chassis strains to be used in a variety of synthetic biology applications. For example, the methods disclosed herein can be used to create chassis strains de novo from two yeast strains with desirable traits, producing a new chassis strain with both desired characteristics without sporulation. Importantly, chassis strains made by the disclosed methods may lack drug resistance cassettes and auxotrophies, both of which are desirable qualities in beverage and biofuel strains.

Methods of making synthetic yeast cells are provided. The methods may be directed to mating together two diploid (or higher ploidy) yeast species or hybrids. The two yeast species or hybrids may be the same or different strains of a yeast species or hybrid or may be different yeast species or hybrids entirely. For example, the methods may include i) introducing a first polynucleotide comprising a first promoter operably connected to an HO polynucleotide encoding a yeast Ho protein, a first selectable marker cassette, and a first yeast origin of replication into a first yeast cell from a first yeast species or hybrid to produce a first transformed yeast cell, ii) introducing a second polynucleotide comprising a second promoter operably connected to an HO polynucleotide encoding a yeast Ho protein, a second selectable marker cassette, and a second yeast origin of replication into a second yeast cell from a second yeast species or hybrid to produce a second transformed yeast cell, and iii) co-culturing the first transformed yeast cell with the second transformed yeast cell to produce a mating mixture.

Alternatively, the methods of the present invention may also be directed to mating a diploid (or higher ploidy) yeast species with a haploid yeast species. The haploid yeast species may be the same or a different yeast strain as the diploid (or higher ploidy) yeast species or may be a different yeast species entirely. Such methods include i) introducing a first polynucleotide comprising a first promoter operably connected to an HO polynucleotide encoding a yeast Ho protein, a first selectable marker cassette, and a first yeast origin of replication into a first yeast cell from a first yeast species or hybrid to produce a first transformed yeast cell, ii) introducing a second polynucleotide comprising a second selectable marker cassette, and a second yeast origin of replication into a second yeast cell from a second haploid yeast species to produce a second transformed yeast cell, and iii) co-culturing the first transformed yeast cell with the second transformed yeast cell to produce a mating mixture.

A “yeast species,” as used herein, may refer to a substantially pure yeast species or to a yeast strain that is a mixed yeast species. A substantially pure yeast species may contain greater than 95%, 96%, 97%, 98%, 99%, or 99.9% chromosomal content from a single yeast species. For example, strains of Saccharomyces cerevisiae that contain greater than 95%, 96%, 97%, 98%, 99%, or 99.9% Saccharomyces cerevisiae chromosomal content may be considered a substantially pure yeast species and would be considered to be a Saccharomyces cerevisiae species. A mixed yeast species may contain substantial chromosomal content from 2 or more different yeast species. For example, a particular yeast strain may have 90% chromosomal content from Saccharomyces uvarum and 5% chromosomal content from Saccharomyces cerevisiae and 5% chromosomal content from Saccharomyces eubayanus. In the case of mixed species, and in accordance with the present invention, the yeast species of a yeast cell from a mixed yeast species is determined by which species contributes the majority of the chromosomal content in the cell. Thus, in the preceding mixed species example, the exemplary yeast strain would be considered a Saccharomyces uvarum species.

The yeast species or hybrids of the present invention may be ascomycetes. Suitable yeast species or hybrids of the present invention may be from the family Saccharomycetaceae. In some embodiments, the yeast species or hybrids are from the genus Saccharomyces. Suitable Saccharomyces species may include, without limitation, Saccharomyces cerevisiae, Saccharomyces paradoxus, Saccharomyces mikatae, Saccharomyces jurei, Saccharomyces arboricola, Saccharomyces kudriavzevii, Saccharomyces uvarum, and Saccharomyces eubayanus. Suitable Saccharomyces hybrids may include, without limitation, Saccharomyces hybrids having at least one parent selected from the group consisting of Saccharomyces cerevisiae, Saccharomyces paradoxus, Saccharomyces mikatae, Saccharomyces jurei, Saccharomyces arboricola, Saccharomyces kudriavzevii, Saccharomyces uvarum, and Saccharomyces eubayanus. Suitable Saccharomyces species may also include any taxonomic synonyms of these species or any newly discovered species to be members of the genus Saccharomyces.

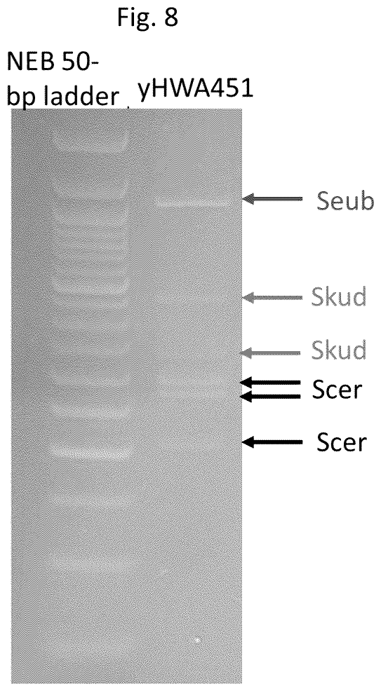

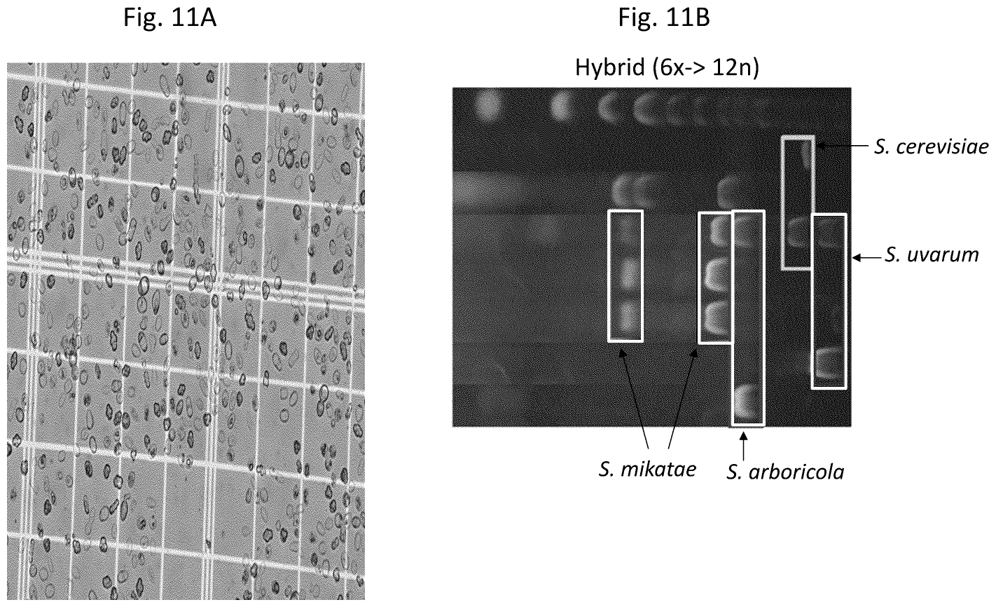

The yeast species of the present methods may be haploid (1N), diploid (2N), triploid (3N), tetraploid (4N), pentaploid (5N), hexaploid (6N), heptaploid (7N), octaploid (8N), nonaploid (9N), decaploid (10N), 11N, 12N, 13N, 14N, 15N, 16N or more. The yeast hybrids of the present methods may be diploid (2N), triploid (3N), tetraploid (4N), pentaploid (5N), hexaploid (6N), heptaploid (7N), octaploid (8N), nonaploid (9N), decaploid (10N), 11N, 12N, 13N, 14N, 15N, 16N or more. In some embodiments, the first yeast species or hybrid and the second yeast species or hybrid of the present methods are diploid. In other embodiments, they may be tetraploid or higher ploidy, such as the embodiment shown to create hexaploids (FIG. 8) and 12N yeasts (FIG. 11B).

Suitable yeast species that may used in accordance with the present invention include, without limitation, species of the genera Candida, Saccharomyces, Kazachstania, Nakaseomyces, Kluyveromyces, Lachancea, Naumovozyma, Vanderwaltozyma, Tetrapisispora, Yueomyces, Zygosaccharomyces, Torulaspora, Zygotorulaspora, Eremothecium, and Ashbya. In general, we expect the present invention to work on genera whose life cycle, mating-type locus, and silent mating cassettes are sufficiently similar to Saccharomyces, especially with respect to Ho protein binding to and cutting the MAT locus to enable mating-type switching.

As used herein, a “yeast hybrid” refers to a yeast cell having at least one set of chromosomes from at least two different yeast species. The present methods may not only be performed using two yeast cells from either the same or different yeast species, but may also be used with yeast hybrids. A yeast hybrid may be a naturally occurring hybrid yeast strain, such as the Saccharomyces cerevisiae X Saccharomyces eubayanus hybrids commonly used to produce lager beers. A yeast hybrid may also be a hybrid yeast strain developed synthetically in the lab using, for example, the methods disclosed herein.

The present methods may be performed through 1, 2, 3, 4, 5, 6, 7, 8, 9, 10, or more rounds of mating. Thus, the polyploid yeast cells created using the present methods may be used as starting materials for performing subsequent rounds of the present methods. In the Examples, the inventors use 2 rounds of mating to produce a hexaploid yeast cell having 2 sets of chromosomes from three different yeast species. The inventors were further able to combine two tetraploid species to generate an 8N yeast and then use this 8N yeast to mate with another 4N yeast to generate a 12N yeast. See, e.g., Example 2 and FIGS. 8-11. The inventors also conjecture that the hexploid yeast cell could be mated with a tetraploid using the methods described herein to produce a decaploid (10N) yeast cell. It thus is particularly envisioned that the present methods may be performed reiteratively to create synthetic yeast strains that have large sets of chromosomes (ploidy) and/or chromosomes or chromosomal segments from multiple yeast species.

In some embodiments of the present methods, the first yeast species or hybrid and the second yeast species or hybrid are different yeast species or hybrids. As used herein, a “different yeast species or hybrid” refers to yeast species or hybrids that are not the same as determined in view of the “yeast species” and “yeast hybrid” definitions provided herein. For example, if the first yeast species or hybrid was a yeast strain having 90% chromosomal content from Saccharomyces uvarum and 5% chromosomal content from Saccharomyces cerevisiae and 5% chromosomal content from Saccharomyces eubayanus and the second yeast species was a substantially pure Saccharomyces uvarum strain, the first yeast species or hybrid and the second yeast species or hybrid would be considered the same—Saccharomyces uvarum. Examples of embodiments where the first yeast species or hybrid and the second yeast species or hybrid would be considered different might include the first yeast species or hybrid being Saccharomyces cerevisiae and the second yeast species or hybrid being Saccharomyces eubayanus.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases may refer to DNA or RNA of natural or synthetic origin (which may be single-stranded or double-stranded and may represent the sense or the antisense strand). Polynucleotides may also include modified nucleotides that permit correct read-through by a polymerase and do not alter expression of, for example, the HO polypeptide encoded by polynucleotides described herein. Preferably, the polynucleotides described herein are DNA.

The polynucleotides of the present invention may further include a promoter. The terms “promoter,” “promoter region,” or “promoter sequence” refer generally to transcriptional regulatory regions of a gene, which may be found at the 5′ or 3′ side of the HO polynucleotide, or within the coding region of the HO polynucleotide. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the promoters within the polynucleotides of the present invention may be operably connected to the HO polynucleotide. As used herein, a promoter is “operably connected to” or “operably linked to” when it is placed into a functional relationship with a second polynucleotide sequence. For instance, a promoter is operably connected to an HO polynucleotide if the promoter is connected to the HO polynucleotide such that it may effect transcription of the HO polynucleotide coding sequence. In various embodiments, the HO polynucleotides may be operably linked to at least 1, at least 2, at least 3, at least 4, at least 5, or at least 10 promoters.

Promoters useful in the practice of the present invention include, but are not limited to, constitutive, inducible, temporally-regulated, and chemically regulated promoters. Preferably, the promoters are inducible. Suitable inducible promoters for expression in yeast include, without limitation, galactose inducible promoters (i.e., GAL1) and doxycycline-inducible promoters. Those of skill in the art are familiar with a wide variety of additional promoters for use in various yeast species.

In the present methods, the first and second promoters may be the same promoter or may be different promoters. In some embodiments, the first and second promoters are the same inducible promoter. In embodiments where the first and second promoters are inducible promoters, the present methods may further include inducing the first and second inducible promoters prior to co-culturing the first transformed yeast cell with the second transformed yeast cell (step (iii) of the present methods).

The polynucleotides of the present invention may include an HO polynucleotide encoding a yeast Ho protein. Yeast Ho proteins are site-specific endonucleases that produce a double-strand break in the MAT locus. The double-strand break is followed by a unidirectional gene conversion event that replaces the information at the MAT locus by information copied from either of the two homologous loci (HMR and HML). Yeast Ho proteins may be any of the Ho proteins found in any yeast species including, without limitation, those yeast species closely related to Candida glabrata. The S. cerevisiae Ho recognition site in MATa (TTTCCGCAACAGT; SEQ ID NO: 13) and MATc (TTCGCGCAACAGT; SEQ ID NO: 14) differs from C. glabrata by one nucleotide, but MATa Ho recognition site is recognized by ScerHo. The presence of the HO locus may extend to all genera classified in the family Saccharomycetaceae because Kluyveromyces lactis has a relic of HO, but it is absent from other species, such as Kluyveromyces waltii. Suitably, the protein sequence of an exemplary yeast Ho protein from Saccharomyces cerevisiae is indicated in SEQ ID NO: 11. In some embodiments, the yeast Ho protein comprises SEQ ID NO: 11 or a mutant, variant, derivative, or fragment thereof.

As used herein, a “protein,” “polypeptide,” or “peptide” may be used interchangeably to refer to a polymer of amino acids. A “protein” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

A yeast Ho protein may include “mutant” proteins, variants, and derivatives thereof. As used herein the term “wild-type” is a term of the art understood by skilled persons and means the typical form of an organism, strain, gene or characteristic as it occurs in nature as distinguished from mutant or variant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a yeast Ho protein mutant or variant protein may have one or more insertions, deletions, or substitution of at least one amino acid residue relative to the yeast Ho “wild-type” protein. The protein sequences of a “wild-type” yeast Ho protein from Saccharomyces cerevisiae is presented as SEQ ID NO: 11. This sequence may be used as a reference sequence.

A yeast Ho protein may be a full-length protein or may be fragments of the full-length protein. As used herein, a “fragment” is a portion of an amino acid sequence, which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference protein, respectively. In some embodiments, a fragment may comprise at least 5, 10, 15, 20, 25, 30, 40, 50, 60, 70, 80, 90, 100, 150, 250, or 500 contiguous amino acid residues of a reference protein. Fragments may be preferentially selected from certain regions of a molecule. The term “at least a fragment” encompasses the full-length polypeptide. A fragment may include an N-terminal truncation, a C-terminal truncation, or both truncations relative to the full-length yeast Ho protein. Preferably, a fragment of a yeast Ho protein includes amino acid residues required for recognition and cleavage of the MAT locus site.

A “deletion” in a yeast Ho protein refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a yeast Ho protein refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant of a yeast Ho protein may have N-terminal insertions, C-terminal insertions, internal insertions, or any combination of N-terminal insertions, C-terminal insertions, and internal insertions.

Regarding proteins, the phrases “percent identity,” “% identity,” and “% sequence identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. As described herein, variants, mutants, or fragments (e.g., a yeast Ho protein variant, mutant, or fragment thereof) may have 99%, 98%, 97%, 96%, 95%, 94%, 93%, 92%, 91%, 90%, 80%, 70%, 60%, or 50% amino acid sequence identity relative to a reference molecule (e.g., relative to a full-length yeast Ho protein (SEQ ID NO: 11)).

Protein sequence identity may be measured over the length of an entire defined protein sequence, for example, as defined by a particular SEQ ID number, or may be measured over a shorter length, for example, over the length of a fragment taken from a larger, defined protein sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The amino acid sequences of the yeast Ho protein variants, mutants, or derivatives as contemplated herein may include conservative amino acid substitutions relative to a reference amino acid sequence. For example, a variant, mutant, or derivative polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The HO polynucleotides encoding the yeast Ho proteins, fragments, variants, mutants, or derivatives thereof may be any polynucleotide encoding the appropriate yeast Ho protein amino acid sequence. Those of skill in the art also understand the degeneracy of the genetic code and that a variety of polynucleotides can encode the same polypeptide. In some embodiments, the polynucleotides may be codon-optimized for expression in a particular yeast cell. While particular nucleotide sequences which are found in Saccharomyces cerevisiae (i.e., SEQ ID NO: 12) are disclosed herein any nucleotide sequences may be used which encode a desired form of the yeast Ho proteins described herein. Thus, non-naturally occurring sequences may be used. These may be desirable, for example, to enhance expression in a particular yeast species. Computer programs for generating degenerate coding sequences are available and can be used for this purpose as well as other means.

The polynucleotides of the present invention may also include a selectable marker cassette. In the Examples, the selectable marker cassettes used in the exemplary HyPr pHCT2 and pHMK34 plasmids conferred resistance to nourseothricin (natMX4) and hygromycin (hphMX), respectively. Two additional plasmids were generated for use in the methods provided herein including pHRW32 (SEQ ID NO: 16), which includes zeocin resistance and pHRW40 (SEQ ID NO: 15), which includes G418 resistance. Other selectable markers conferring resistance to other antibiotics such as, kanMX, amdS, TK, Sh ble or ble, which confer resistance to G418, fluoroacetamide, antifolates, Zeocin or phleomycin, respectively could also be used. Those of skill in the art will appreciate that additional combinations of selectable markers can be used as well. Other forms of selectable markers may be used such as markers that provide a growth advantage or colorimetric selection other than antibiotic resistance. The selectable marker cassettes include a polynucleotide encoding the selectable marker operably connected to a promoter capable of inducing transcription of the selectable marker.

Suitably, the first selectable marker cassette and the second selectable marker cassette encode different selectable markers. By encoding different complementary selectable marker cassettes, the disclosed methods readily select for mating events between the first yeast cell and the second yeast cell.

The polynucleotides of the present invention may also include a yeast origin of replication to allow replication of the polynucleotides in a particular yeast species. Suitably, the first yeast origin of replication and the second yeast origin of replication are functional across many yeast species including, without limitation, all Saccharomyces species. In the Examples, the yeast origin of replication used in the exemplary HyPr pHCT2 and pHMK34 plasmids included KARS101 from Kluyveromyces lactis and S. cerevisiae CEN and ARS sequences to improve stability in multiple yeast species.

As used herein, “introducing” describes a process by which exogenous polynucleotides (e.g., DNA or RNA) are introduced into a recipient yeast cell. Methods of introducing polynucleotides into a yeast cell are known in the art and may include, without limitation, transformation methods such as electroporation and lithium acetate/single-stranded carrier DNA/PEG methods. Transformation may occur under natural or artificial conditions according to various methods well known in the art and may rely on any known method for the insertion of foreign nucleic acid sequences into a yeast cell. The method for transformation may be selected based on the type of yeast cell being transformed and may include, but is not limited to, electroporation, heat shock, chemical transformation methods such as lithium acetate/single-stranded carrier DNA/PEG methods.

As used herein, a “transformed” yeast cell refers to a yeast cell carrying an exogenous polynucleotide that was introduced into the yeast cell using the transformation methods described herein.

As used herein, “co-culturing” refers to growing at least two yeast cells in an appropriate media. Common yeast growth media are well known in the art and include, without limitation, YPD media. In some embodiments, the present methods may include co-culturing the first transformed yeast cell with the second transformed yeast cell to produce a mating mixture. A “mating mixture” refers to a mixture of at least two yeast cells, which will likely have at least one yeast cell having a MATa mating type and at least one yeast cell having a MATa mating type. Suitably, in some embodiments, the mating mixture is introduced onto plates to facilitate mating of the yeast cells.

In some embodiments, the present methods may further include selecting at least a portion of the mating mixture for the first selectable marker and the second selectable marker to select for a synthetic yeast cell. A “synthetic yeast cell” refers to a yeast that may be made using the present methods. The “selecting at least a portion of the mating mixture” may be performed by simply adding the appropriate agents (i.e., antibiotics) to the growth media to select for cells harboring the first selectable marker and/or the second selectable marker.

The present methods may further include culturing the synthetic yeast cell in non-selective media to produce a synthetic yeast cell lacking the first polynucleotide and/or the second polynucleotide. Such embodiments of the present methods may provide an efficient route towards meeting the Generally Recognized As Safe (GRAS) standards of the United States Department of Agriculture and Food and Drug Administration.

In some embodiments, the present methods may further include selecting the synthetic yeast cell under conditions to optimize the synthetic yeast cell for a fermentation process. As used herein, a “fermentation process” may involve any fermentation process involved in beer making, wine making, cider, sake, and traditional alcoholic beverages (mezcal, chicha, mudai, among others), biofuel production, or other commercially valuable process. Extensive prior work has shown that yeast polyploid strains, for example, are relatively unstable, rapidly losing chromosomes to form aneuploid strains. This loss occurs rapidly with genome content reduction to near-diploid levels in 200 to 800 generations. The inventors expect that synthetic yeast cells made via the methods disclosed herein to behave in a similar manner. This could, in fact, be a desirable trait for many applications, as placing an unstable polyploid strain in a selective condition will influence which components of the genome are retained or lost from which parent, allowing for more rapid adaptation to that condition. The inventors expect that synthetic yeast cells made by the methods disclosed herein will also evolve aneuploidy given enough time, but again, many of these aneuploidies may be advantageous in the conditions where they are evolved.

Synthetic yeast cells are provided. The synthetic yeast cells may include 5, 6, 7, 8, 9, 10, 11, 12, 13, 14, 15, 16, or more sets of yeast chromosomes. As used herein, a “set of chromosomes” refers to a complete or substantially complete set of chromosomes. The sets of chromosomes may all come from the same yeast species or may come from 2, 3, 4, 5, 6, 7, 8, or more different yeast species.

The synthetic yeast cells of the present invention may also include chromosomes or chromosomal segments from 3, 4, 5, 6, 7, 8, or more yeast species.

The synthetic yeast cells may be an ascomycete including, without limitation, a yeast species from the family Saccharomycetaceae or a Saccharomyces. In some embodiments, the chromosomes or chromosomal segments in the synthetic yeast cells are from an ascomycete, including, without limitation, Saccharomyces. Suitable Saccharomyces species include, without limitation, Saccharomyces cerevisiae, Saccharomyces paradoxus, Saccharomyces mikatae, Saccharomyces jurei, Saccharomyces arboricola, Saccharomyces kudriavzevii, Saccharomyces uvarum, and Saccharomyces eubayanus.

The synthetic yeast cells may be selected from the strains consisting of yHWA358, yHWA375, yHWA377, yHWA425, yHWA439, yHWA451, yHWA452, yHWA455, and yHWA456, yHWA457, yHWA458, yHWA459, yHWA460, any of the strains identified in Table 1 or any other yeast strain developed using the methods provided herein.

Methods of using the synthetic yeast cells described herein are provided. The methods of use may include using any one of synthetic yeast cells described herein or any one of the synthetic yeast cells made by the methods disclosed herein in a fermentation process. In some embodiments, the fermentation process may be involved in beer making, wine making, biofuel production, or another commercially valuable process.

Kits are provided. The kits may include a first polynucleotide comprising a first promoter operably connected to an HO polynucleotide encoding a yeast Ho protein, a first selectable marker cassette, and a first yeast origin of replication, and a second polynucleotide comprising a second promoter operably connected to an HO polynucleotide encoding a yeast Ho protein, a second selectable marker cassette, and a second yeast origin of replication. In some embodiments, the first selectable marker cassette and the second selectable marker cassette encode different selectable markers.

Optionally, the first promoter and/or the second promoter may be an inducible promoter. The first yeast origin of replication and the second yeast origin of replication may also be functional across all Saccharomyces species or other yeast species.

The kits may further include nourseothricin, hygromycin, G418, fluoroacetamide, antifolates, Zeocin, phleomycin, doxycycline, at least two different Saccharomyces species and/or instructions for performing any one of the methods of disclosed herein.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—Efficient Engineering of Marker-Free Synthetic Allotetraploids of Saccharomyces

Saccharomyces interspecies hybrids are critical biocatalysts in the fermented beverage industry, including in the production of lager beers, Belgian ales, ciders, and cold-fermented wines. Current methods for making synthetic interspecies hybrids are cumbersome and/or require genome modifications. We have developed a simple, robust, and efficient method for generating allotetraploid strains of prototrophic Saccharomyces without sporulation or nuclear genome manipulation. S. cerevisiae x S. eubayanus, S. cerevisiae x S. kudriavzevii, and S. cerevisiae x S. uvarum designer hybrid strains were created as synthetic lager, Belgian, and cider strains, respectively. The ploidy and hybrid nature of the strains were confirmed using flow cytometry and PCR-RFLP analysis, respectively. This method provides an efficient means for producing novel synthetic hybrids for beverage and biofuel production, as well as for constructing tetraploids to be used for basic research in evolutionary genetics and genome stability.

Many eukaryotic organisms are able to reproduce sexually, wherein meiosis serves to both increase genetic diversity and to repair genetic material (Kohl and Sekelsky, 2013). As with other eukaryotes, fungal meiosis generally initiates in diploid cells (John, 1990). Regulation of this process involves specialized genetic loci called the mating-type (MAT) loci (Kronstad and Staben, 1997). While basidiomycetes may possess thousands of different mating types, ascomycetes possess only two (Casselton and Olesnicky, 1998). Ascomycete MAT loci contain between one and three genes. The mating-type genes are not recently diverged homologs, and are thus described as idiomorphs, rather than alleles. These mating-type genes encode transcription factors that regulate the expression of meiosis-specific genes (Van Heeckeren et al., 1998), as well as other genes that function in self recognition (Glass et al., 2000), pheromone production (Bardwell, 2004), and non-homologous end joining (Frank-Vaillant and Marcand, 2001). Heterothallic ascomycetes, such as Neurospora crassa, require two individuals of different mating types to contribute haploid nuclei towards a transient diploid meiotic precursor cell (Glass et al., 1990; Staben and Yanofsky, 1990).

The genus Saccharomyces is composed of unicellular diploid ascomycete fungi (Hittinger, 2013). The S. cerevisiae MAT idiomorphs are termed MATa and MA Td, and both possess two genes (Haber, 2012). Unlike many other ascomycetes where vegetative haploid nuclei give rise to a transient diploid cell, Saccharomyces haploid spores of a single mating type rapidly give rise to stable diploid cells possessing both mating-type loci by way of mating-type switching (Haber, 2012). When a newly germinated haploid cell divides, the mother cell expresses HO, producing an endonuclease that specifically targets the mating-type locus. The resulting DNA double-strand break (DSB) is repaired using information from silenced copies of either mating-type idiomorph found on the same chromosome as the MAT locus (designated HML and HMR). Repairing the DSB lesion occurs by replacing the existing MAT idiomorph with the silenced copy of the other MAT idiomorph, resulting in the mother cell switching to the opposite mating type. As the daughter cell retains the initial idiomorph, mating-type switching ensures two haploid cells of opposite mating-types are adjacent to one another for schmooing and the formation of a diploid zygote. Deletion of HO prevents mating types from being switched, forming stable haploid strains, such as those typically used in laboratory research (Walker et al., 2003). Indeed, the plasticity of Saccharomyces ploidy is a major component of the awesome power of yeast genetics. Induced HO expression from plasmids in hoi strains triggers mating-type switching, resulting in diploidization of otherwise stable haploid strains (Herskowitz and Jensen, 1991; Jensen and Herskowitz, 1984). This diploid yeast cell can then be sporulated by standard methods, dissected, and the progeny screened for their mating type. The resulting haploids are isogenic to the original haploid strain save for their mating types, which can then be used in downstream genetic crosses. In addition to long-standing use to control mating type and ploidy in lab strains of S. cerevisiae, induction of HO expression has also been used to enable crosses between a fertile strain of S. uvarum and a sterile strain whose genome was predominantly S. uvarum, allowing a trait to be mapped using standard meiotic techniques (Schwartz et al., 2012).

All seven known species of Saccharomyces yeasts possess the same mating-type locus organization and a predominantly diploid or diplontic lifestyle. Only limited pre-zygotic speciation barriers exist between Saccharomyces species (Maclean and Greig, 2008), making hybridization a trivial process with marked heterothallic haploids (Bullard et al., 2010; Hebly et al., 2015; Hittinger, 2013; Piotrowski et al., 2012; Scannell et al., 2011; Swain Lenz et al., 2014; Tirosh et al., 2009). Hybridization events also happen in the wild, though at a very low frequency (Mortimer, 2000). These hybrids arise when a cell of one species mates with a cell from another. Interspecies hybridization can either occur between two haploids, as is typically done in the laboratory, or through “rare mating” when one or both parents are diploid. Rare mating diploids can gain mating competency by simply inactivating one idiomorph to become hemizygous or undergoing spontaneous gene conversion at the MAT locus and becoming MATa/MATa or MATα/MATα diploids (Gunge and Nakatomi, 1972).

Interspecies hybrids spontaneously arising in this manner have found purchase in the conditions created by humans during industrial fermentations, such as brewing; indeed, hybrids produce many commercially important fermented beverages. For example, Saccharomyces cerevisiae x Saccharomyces eubayanus hybrids are used to produce lager beer, the most common fermented beverage on the planet (Corran, 1975; Libkind et al., 2011). Two major lineages are used in lager production (Dunn and Sherlock, 2008; Nakao et al., 2009; Walther et al., 2014), and recent evidence indicates that these two lineages arose from independent hybridization events, suggesting that genetic diversity from the parental populations may be one contributor to the phenotypic differences seen in modern industrial strains (Baker et al., 2015). Many other fermented beverages also make use of Saccharomyces hybrids: S. cerevisiae x Saccharomyces uvarum hybrids are used in production of some ciders and cold-fermented wines (Le Jeune et al., 2007; Masneuf et al., 1998; Pérez-Través et al., 2014b), while many Belgian ale and some European wine yeasts are S. cerevisiae x Saccharomyces kudriavzevii hybrids (Peris et al., 2012a, 2012b).

The discovery of the wild genetic stocks related to the constituent species of industrial interspecies hybrids (Almeida et al., 2014; Libkind et al., 2011; Sampaio and Gonçalves, 2008), as well as the discovery of several more divergent lineages that do not seem to have contributed to the production of fermented beverages (Bing et al., 2014; Hittinger et al., 2010; Leducq et al., 2014; Liti et al., 2009, 2005; Peris et al., 2014; Wang et al., 2012), has raised interest in synthetic interspecies hybrids that may possess novel properties and allow for strain improvement. New hybrid brewing strains have been generated by a laborious process of isolating auxotrophic mutants, which arise spontaneously at low frequency, followed by crossing to obtain hybrids (Krogerus et al., 2015; Pérez-Través et al., 2012). Although this method lacks markers, which would likely streamline approval for food and beverage applications, the strains contain mutations in important biosynthetic pathways. An easier method is to first generate stable heterothallic haploids for one or both parents, for example by replacing HO with drug markers, followed by interspecies crosses (Bullard et al., 2010; Swain Lenz et al., 2014; Tirosh et al., 2009). Variations of this strategy have used complementary drug markers and auxotrophic mutants in one species and spore dissection of wild-type diploids from another (Hebly et al., 2015; Piatkowska et al., 2013). However, the persistence of drug markers in the latter hybrids raises legitimate concerns about their safety that would need to be addressed prior to introducing them into the food and beverage industry.

Here we describe a generalized method for the efficient production of designer hybrid strains of Saccharomyces based on a series of inducible expression plasmids named HyPr (for Hybrid Production). These plasmids contain complementary dominant drug-resistance markers, a doxycycline-inducible HO cassette, and a generalized replication origin that provides functionality across Saccharomyces and many other yeasts. HyPr efficiently produces allotetraploid and autotetraploid strains of Saccharomyces, as well as allohexaploid strains and strains of higher ploidies. The resulting strains can be rapidly screened for plasmid loss, providing an efficient route towards meeting the standards that the United States Department of Agriculture and Food and Drug Administration have previously applied to a wine strain of S. cerevisiae, which is Generally Recognized As Safe (GRAS) (Husnik et al., 2006). These techniques also provide a valuable and general research tool for basic and applied research on prototrophic hybrids and polyploids of Saccharomyces.

Materials and Methods Strains, culture conditions, and media Strains used in this work are found in Table 1. S. cerevisiae strains were cultured at 30° C., except when they were being co-cultured with another species. All other Saccharomyces species were cultured at room temperature (22-23° C.). Routine cultures were maintained in YPD (1% yeast extract, 2% peptone, 2% glucose). Hygromycin was added to YPD at a concentration of 200 mg/L to make YPD +hyg. Nourseothricin was added to YPD at a concentration of 100 mg/L to make YPD +nat. All liquid media was solidified when needed by the addition of 1.8% agar.

TABLE 1
Strains used in this work

| Identifier | Species | Genotype | Source |
|---|---|---|---|
| RM11-1a | Saccharomyces cerevisiae | MATa leu2-Δ ura3-Δ hoΔ::KanMX | Brem et al. (2002) |
| Ethanol Red | Saccharomyces cerevisiae | MATa/MATα | Fermentis |
| NRRL YB-210 | Saccharomyces cerevisiae | MATa/MATα | Mortimer and Johnston |
| | | | (1986) |
| Wyeast #1007, | Saccharomyces cerevisiae | MATa/MATα | Wyeast |
| German Ale | | | |
| ZP 591 | Saccharomyces kudriavzevii | MATa/MATα | Sampaio and Gonçalves |
| | | | (2008) |
| CBS 7001 | Saccharomyces uvarum | MATa/MATα | Scannell et al. (2011) |
| yHKS210 | Saccharomyces eubayanus | MATa/MATα | Peris et al. (2014) |
| White Labs WLP830, | Saccharomyces cerevisiae × | MATa/MATa/MATα/MATα | White Labs |
| German Lager, | Saccharomyces eubayanus | | |
| Weihenstephan 34/70 | | | |
| yHWA338 | Saccharomyces cerevisiae | RM11-1a [pHCT2] | This work |
| yHWA340 | Saccharomyces cerevisiae | Ethanol Red [pHCT2] | This work |
| yHWA341 | Saccharomyces cerevisiae | NRRL YB-210 [pHMK34] | This work |
| yHWA348 | Saccharomyces uvarum | CBS 7001 [pHMK34] | This work |
| yHWA350 | Saccharomyces cerevisiae | Wyeast #1007 [pHCT2] | This work |
| yHWA352 | Saccharomyces eubayanus | yHKS210 [pHMK34] | This work |
| yHWA354 | Saccharomyces kudriavzevii | ZP 591 [pHMK34] | This work |
| yHWA358 | Saccharomyces cerevisiae | Ethanol Red [pHCT2] × NRRL YB-210 | This work |
| | | [pHMK34] | |
| yHWA375 | Saccharomyces cerevisiae × | Wyeast #1007 [pHCT2] × ZP 591 | This work |
| | Saccharomyces kudriavzevii | [pHMK34] | |
| yHWA377 | Saccharomyces cerevisiae × | Wyeast #1007 [pHCT2] × CBS 7001 | This work |
| | Saccharomyces uvarum | [pHMK34] | |
| yHWA425 | Saccharomyces cerevisiae × | Wyeast #1007 [pHCT2] × yHKS210 | This work |
| | Saccharomyces eubayanus | [pHMK34] | |
| yHWA439 | Saccharomyces cerevisiae × | Wyeast #1007 × yHKS210 | This work |
| | Saccharomyces eubayanus | | |

| Identifier | Species | Genotype | Source/Comments |
|---|---|---|---|
| yHWA 451 | S. cerevisiae/ | MATa/MATa/MATa/ | putative hexaploid with PCR-RFLP |
| | S. eubayanus/ | MATα/MATα/MATα | confirmed Scer, Seub, and Skud |
| | S. kudravzevii | [pHCT2][pHMK34] | genomes present; grown on SCGly |
| | | | to confirm respiration capability |
| yHWA 452 | S. cerevisiae/ | MATa/MATa/MATa/ | putative hexaploid with PCR-RFLP |
| | S. eubayanus/ | MATα/MATα/MATα | confirmed Scer, Seub, and Skud |
| | S. kudravzevii | [pHCT2][pHMK34] | genomes present; grown on SCGly |
| | | | to confirm respiration capability |
| yHWA 453 | S. mikatae | MATa/MATα [pHCT2] | Contains pHCT2, transformed by |
| | | | and obtained from Ryan Moriarty |
| yHWA 454 | S. arboricola | MATa/MATα [pHMK34] | Contains pHMK34, transformed by |
| | | | and obtained from Ryan Moriarty |
| yHWA 455 | S. cerevisiae/ | MATa/MATa/MATa/ | putative hexaploid hybrid between |
| | S. eubayanus/ | MATα/MATα/MATα | yHWA450 and yHWA354, presence |
| | S. kudriavzevii | | of Scer Skud and Seub genomes |
| | | | confirmed with PCR-RFLP; cured of |
| | | | both plasmids |
| yHWA 456 | S. cerevisiae/ | MATa/MATa/MATa/ | putative hexaploid hybrid between |
| | S. eubayanus/ | MATα/MATα/MATα | yHWA450 and yHWA354, presence |
| | S. kudriavzevii | | of Scer Skud and Seub genomes |
| | | | confirmed with PCR-RFLP; cured of |
| | | | both plasmids |
| yHWA 457 | S. cerevisiae/ | MATa/MATa/MATa/ | putative hexaploid hybrid between |
| | S. eubayanus/ | MATα/MATα/MATα | yHWA450 and yHWA354, presence |
| | S. kudriavzevii | [pHCT2] | of Scer Skud and Seub genomes |
| | | | confirmed with PCR-RFLP |
| yHWA 458 | S. cerevisiae/ | MATa/MATa/MATa/ | putative hexaploid hybrid between |
| | S. eubayanus/ | MATα/MATα/MATα | yHWA450 and yHWA354, presence |
| | S. kudriavzevii | [pHCT2] | of Scer Skud and Seub genomes |
| | | | confirmed with PCR-RFLP |
| yHWA 459 | S. cerevisiae/ | MATa/MATa/MATa/ | putative hexaploid hybrid between |
| | S. eubayanus/ | MATα/MATα/MATα | yHWA450 and yHWA354, presence |
| | S. kudriavzevii | [pHMK34] | of Scer Skud and Seub genomes |
| | | | confirmed with PCR-RFLP |
| yHWA 460 | S. cerevisiae/ | MATa/MATa/MATa/ | putative hexaploid hybrid between |
| | S. eubayanus/ | MATα/MATα/MATα | yHWA450 and yHWA354, presence |
| | S. kudriavzevii | [pHMK34] | of Scer Skud and Seub genomes |
| | | | confirmed with PCR-RFLP |
| yHRVM 349 | S. cerevisiae | | yHAB335; Nat resistant |
| yHRVM 346 | S. paradoxus | | CBS432; Hyg resistant |
| yHRVM 359 | S. arboricola | | CBS 10644; Nat resistant |
| yHRVM 356 | S. uvarum | | yHCT77; Hyg resistant |
| yHDPN 405 | S. mikatae | | IFO1815; Hyg resistant |
| yHRVM 371 | S. kudriavzevii | | yHAB407; Nat resistant |
| yHRVM 456 | S. cerevisiae × | | yHAB335 × CBS432; Nat + Hyg |
| | S. paradoxus | | resistant; confirmed by RFLP |
| yHRVM 461 | S. uvarum × | | yHCT77 × CBS 10644; Nat + Hyg |
| | S. arboricola | | resistant; confirmed by RFLP |
| yHRVM 501 | S. mikatae × | | IFO1815 × yHAB407; Nat + Hyg |
| | S. kudriavzevii | | resistant; confirmed by RFLP |
| yHRVM 495 | S. cerevisiae × | | yHRVM491 (=yHRVM456 without |
| | S. paradoxus × | | [HYG]) × yHRVM492 |
| | S. uvarum × | | (=yHRVM461 without [NAT]); |
| | S. arboricola | | confirmed by RFLP |
| yHRVM 548 | S. cerevisiae × | | yHRVM534 (=yHRVM501 without |
| | S. paradoxus × | | [HYG]) × yHRVM540 |
| | S. uvarum × | | (=yHRVM495 without [NAT]); |
| | S. arboricola × | | confirmed by RFLP |
| | S. mikatae × | | |
| | S. kudriavzevii | | |

### Saccharomyces Transformation

Transformation of yeast strains was done using the lithium acetate/PEG-4000/carrier DNA method as previously described (Gietz and Woods, 2002) with the following modifications: S. cerevisiae was heat shocked at 42° C. for 30 minutes, S. uvarum and S. eubayanus were heat shocked at 37° C. for 30 minutes, and S. kudriavzevii was heat shocked at 34° C. for 30 minutes. Cells were suspended in YPD, followed by incubation at optimal temperature for three hours before being plated to selective media.

### PCR

Primers used in this work are found in Table 2. Plasmid components were amplified using the Phusion PCR Kit (New England Biolabs, Ipswich, MA) as directed by the product insert. Mating-type screening and PCR-RFLP analysis used the Standard Taq Polymerase (New England Biolabs, Ipswich, MA) system as directed by the product insert for amplification.

TABLE 2
Oligonucleotides used in this work.

| Name | Sequence | Use |
|---|---|---|
| MATa F | CTCCACTTCAAGTAAGAGTTTGGGT | mating type screening |
| | (SEQ ID NO: 1) | |
| MATalpha F | TTACTCACAGTTTGGCTCCGGTGT | mating type screening |
| | (SEQ ID NO: 2) | |
| Common MAT R | GAACCGCATGGGCAGTTTACCTTT | mating type screening |
| | (SEQ ID NO: 3) | |
| oHDP022 | TGATTATAGCCACGGGTGARATGTTYT | amplifies fragment of BRE5 for PCR- |
| | (SEQ ID NO: 4) | RFLP analysis |
| oHDP023 | TGATTATAGCCACKGGTGARATGTTTT | amplifies fragment of BRE5 for PCR- |
| | (SEQ ID NO: 5) | RFLP analysis |
| oHDP024 | TTCAKTCATCAAYTTTGAGGCCCATGT | amplifies fragment of BRE5 for PCR- |
| | (SEQ ID NO: 6) | RFLP analysis |
| oHWA230 | AAACGCTCCCCTCACAGACG | amplifies MX-driven drug markers for |
| | (SEQ ID NO: 7) | marker exchange |
| oHWA231 | CTGGGCAGATGATGTCGAGG | amplifies MX-driven drug markers for |
| | (SEQ ID NO: 8) | marker exchange |
| CTH993 | caaatacacacactaaattaccggatcaattcgg | amplifies S. cerevisiae HO from YCp50- |
| | gggaAAAATGCTTTCTGAAAACACGA | HO-D6, clones into pBM5155 over NotI |
| | (SEQ ID NO: 9) | site by gap repair |
| CTH994 | cctccacctccaccgttaattaacccggggatcc | amplifies S. cerevisiae HO from YCp50- |
| | gGCAGATGCGCGCACCT | HO-D6, clones into pBM5155 over NotI |
| | (SEQ ID NO: 10) | site by gap repair |

Construction of pBM5155, pHCT2, and pHMK34

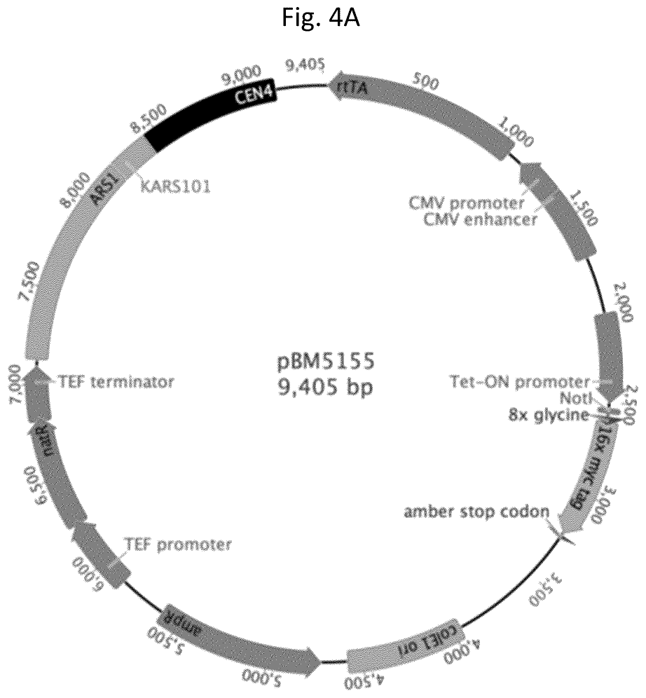

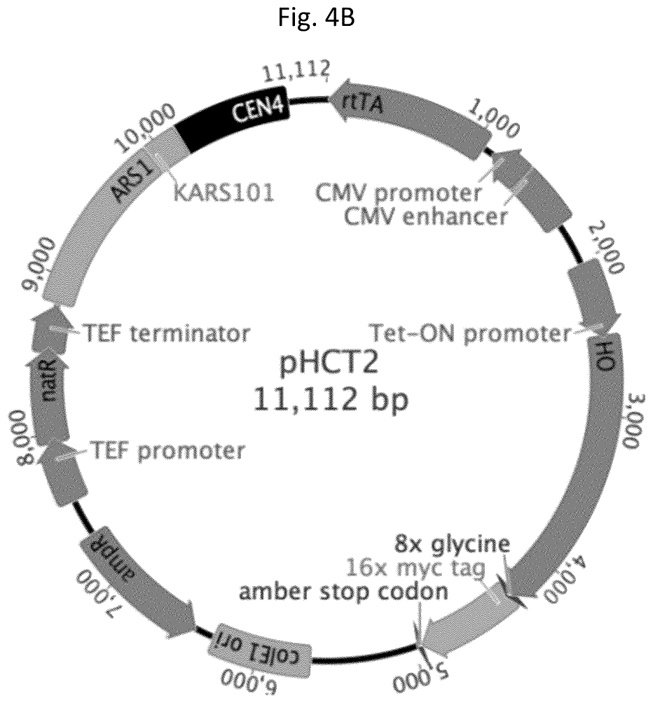

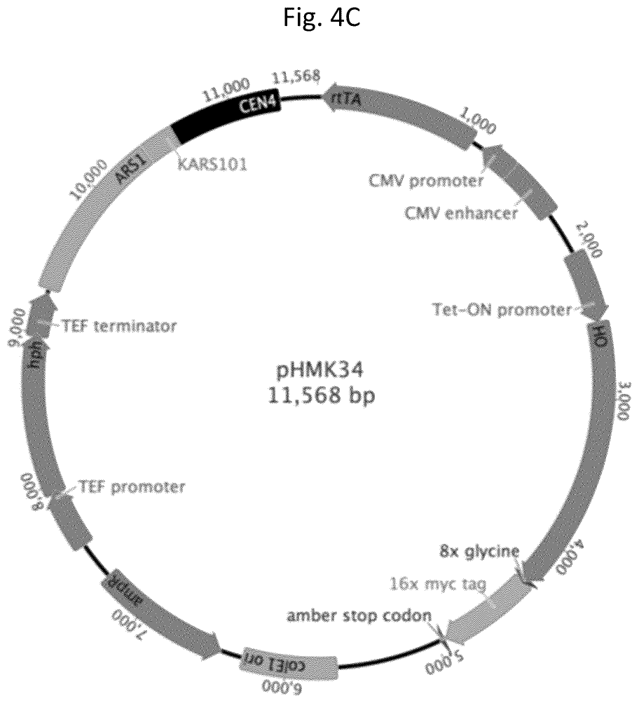

pBM5155 (FIG. 4A) has been previously published and shown to facilitate the doxycycline-inducible gene expression in S. cerevisiae (Sabina and Johnston, 2009), but its sequence and the details of its construction by co-author CTH were not previously documented. pBM5155 was built using the backbone of pCM186 (Gari et al., 1997), which is a CEN plasmid containing all of the heterologous machinery for doxycycline-inducible expression of genes that are cloned into its NotI site, through several consecutive rounds of gap repair cloning in S. cerevisiae, followed by recovery and amplification in Escherichia coli by selection on LB +carbenicillin (or +ampicillin). Modifications of parts were introduced during PCR using oligonucleotides. The original rtTA transcription factor of pCM186, which is driven by a cytomegalovirus promoter, was excised and replaced with rtTA-M2 (Urlinger et al., 2000). The URA3 marker was replaced with a natMX4 marker (Goldstein and McCusker, 1999). An 8× glycine linker was added immediately downstream of the doxycline-inducible promoter and immediately upstream of a 16×myc tag created from a 13×myc tag (Longtine et al., 1998) plus a 3×myc duplication created during gap repair. To improve stability in other yeast species, KARS101 from Kluyveromyces lactis (Fabiani et al., 2001) was added in addition to the S. cerevisiae CEN and ARS sequences already present. Modifications were made sequentially and confirmed by Sanger sequencing. The complete sequence of pBM5155 has been deposited in GenBank under accession KT725394.

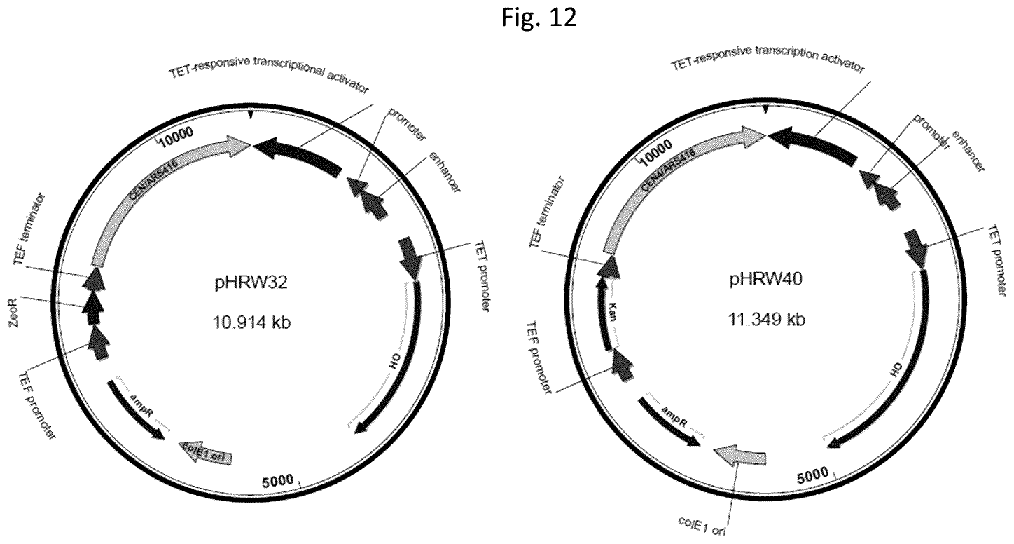

pHCT2 (FIG. 4B) was built by PCR-amplifying S. cerevisiae HO using YCp50-HO-D6 (Russell et al., 1986) as a template, co-transforming that fragment with NotI-digested pBM5155 into S. cerevisiae, selecting on YPD +nat media, and recovering the resulting plasmid into E. coli. The natMX gene in pHCT2 was switched to the hphMX gene (Goldstein and McCusker, 1999) by amplifying the hph gene with oHWA230 and oHWA231, co-transforming the PCR product with AgeI-digested pHCT2 into S. cerevisiae, and selecting on YPD +hyg media. The resulting plasmid was recovered from yeast into bacteria, yielding pHMK34 (FIG. 4C). The sequence of pHCT2 was confirmed by 36-bp single-end Illumina sequencing, followed by assembly with VELVET (Zerbino and Birney, 2008). The manipulated region of pHMK34 was confirmed by PCR analysis and drug resistance. pHCT2 and pHMK34 sequences were deposited in GenBank under the accession numbers KT725395 and KT781077, respectively. Two additional plasmids, pHRW32 and pHRW40 with maps shown in FIG. 12 and sequences provided in SEQ ID NOs: 15 and 16 were also generated and have zeocin and G418 resistance to allow for further selection options. The pHRW40 plasmid is generally selected for on YPD plates with 400 ug/mL G418. The pHRW32 plasmid is generally selected for on YPD plates with 100 ug/mL Zeocin. In some cases, including for pHCT2 and pHMK34, drug concentrations must be optimized for the strains and species being used.

### Induction of HO Expression and Mating

Plasmid-bearing cells were grown to saturation over 12-36 hours in 3 mL liquid YPD +drug. 1.5 mL of culture was discarded and replaced with 1.5 mL fresh liquid YPD +drug, and 3 L of 10 mg/mL of filter-sterilized doxycycline dissolved in water was added. The culture was incubated for four hours to induce HO expression. Induced cells were pelleted, washed with YPD, and 5 μL each of two separate induced cultures were mixed in an Eppendorf tube and patched to a small area of a YPD agar plate to allow mating between newly formed MATa/MATa or MATα/MATα diploids. After 12-36 hours of incubation, a small amount of the patch was struck to or plated on YPD +hyg +nat agar.

### Determination of Ploidy Via Flow Cytometry

Overnight cultures of putative tetraploids were used to inoculate fresh YPD. These cultures were grown until cultures reached exponential phase, then fixed with 70% ethanol and stained with SYBR Green (Thermo Fisher Scientific, Waltham, MA) as previously described (Fortuna et al., 1997); due to possible degradation issues encountered with the normal fixation protocol, the NRRL YB-210 and Wyeast #1007 strains were not heated, nor were they treated with Proteinase K. DNA content was determined with a Guava easyCyte (EMD Millipore, Darmstadt, Germany). Data were processed, analyzed, and visualized in R 2.14.

Confirmation of Hybridization with PCR-Based RFLP

Putative tetraploid hybrids had their genomic DNA extracted, and BRE5 was amplified using primers oHDP022 (Gonzilez et al., 2008), oHDP023, and oHDP024. Resulting PCR products were digested with HaeII (New England Biolabs, Ipswich, MA). Undigested PCR products were visualized on a 1.5% agarose gel, while digested PCR products were visualized on a 3% agarose gel.

### Results

### Doxycycline-Inducible Expression of HO in Saccharomyces

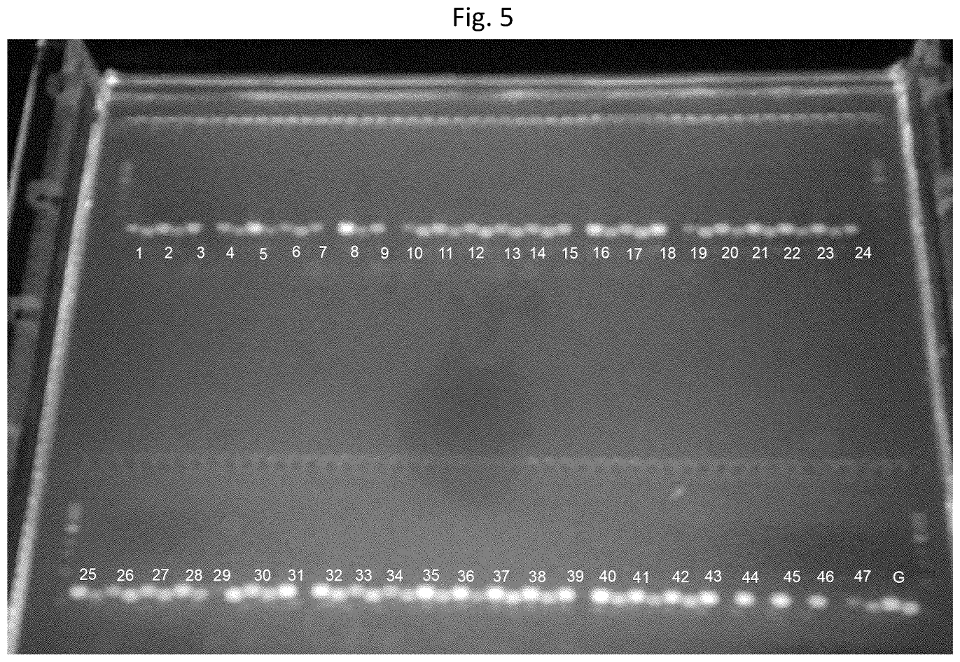

pHCT2 was transformed into the stable haploid S. cerevisiae strain RM11-1a, and HO expression was induced with doxycycline. The induced culture was struck onto YPD +nat plates, and 47 colonies were screened by PCR of the mating-type locus (Gerstein et al., 2006); the Wyeast #1007 German Ale strain was included as a diploid control. 35 colonies produced bands consistent with the presence of both mating-type loci, while 12 colonies only produced one band from primers targeting MATa, the original mating type. The presence of both MATa and MATα are characteristic of a diploid cell, indicating that successful induction of HO by doxycycline, followed by mother-daughter or clonemate selfing, had occurred in the cell lineage to give rise to approximately 75% of the colonies (FIG. 5). Since HO was applied for an extended period of time during this experiment, rather than the normal tightly regulated process, it is formally possible (although highly unlikely) that a fraction of the remaining 25% of the cells could be MATa/MATa diploids rather than haploids.

Intraspecies Tetraploids are Selectable from Co-Cultured, HO-Induced Diploids

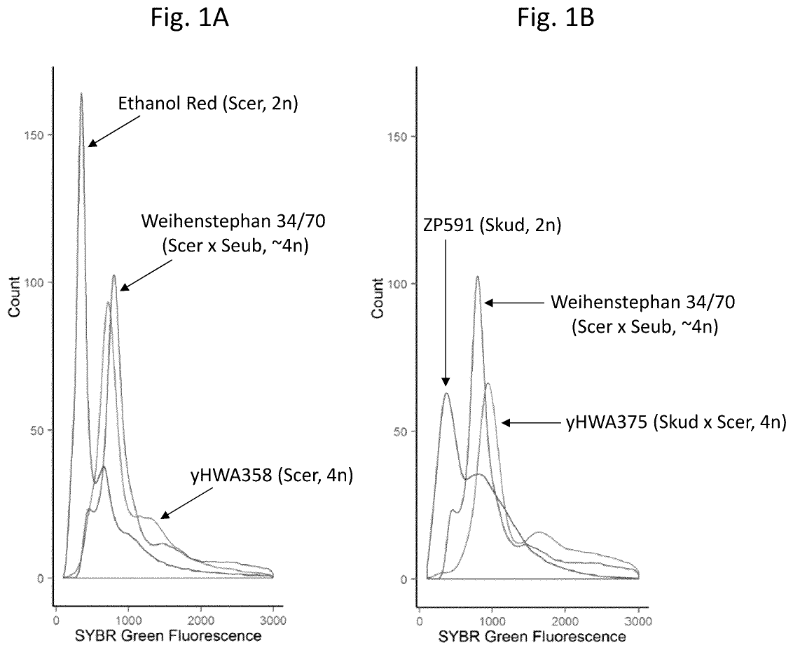

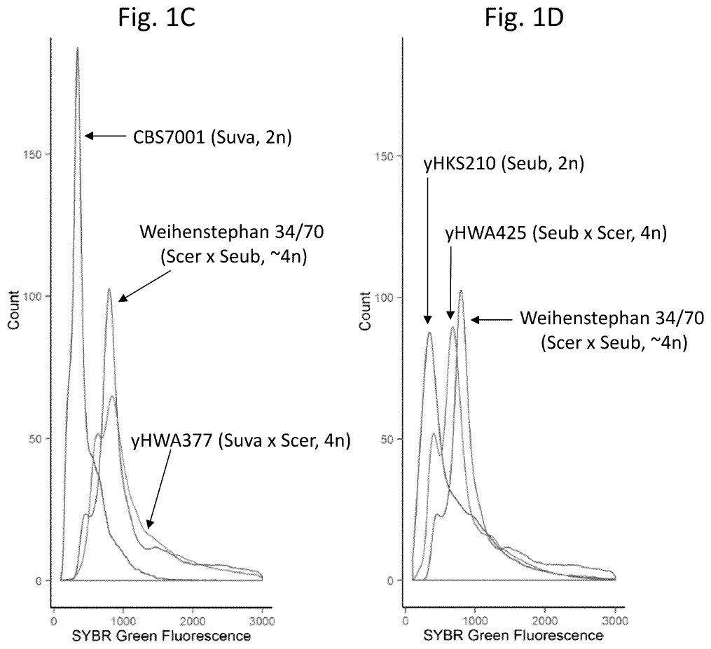

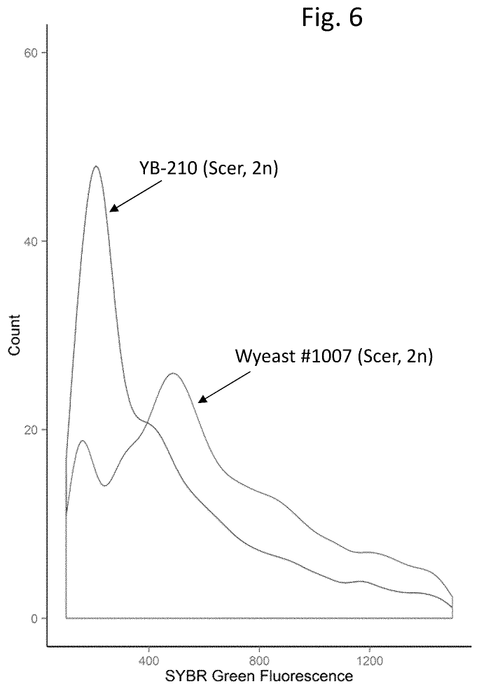

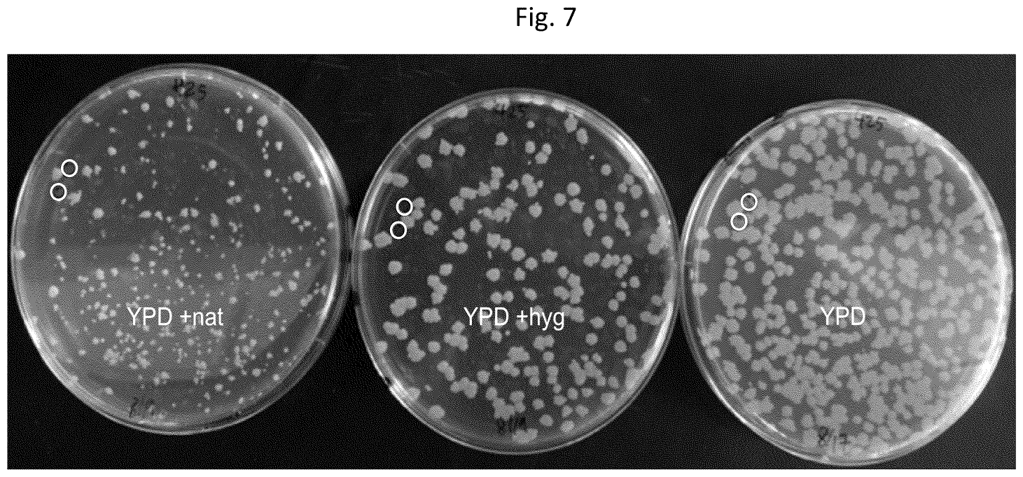

S. cerevisiae strains NRRL YB-210 and Ethanol Red are of biofuel interest due to their natural stress tolerance and routine use in ethanol production, respectively (Wohlbach et al., 2014). Strains containing pHMK34 and pHCT2 (Table 1), respectively, had HO expression induced with doxycycline and were co-cultured on YPD overnight at 30° C. The co-cultured patch was suspended in liquid YPD, and a 10⁻⁴ dilution was made with fresh YPD. 200 μL of dilution was plated onto each of three YPD +hyg +nat plates, while 2 μL was plated onto each of three YPD plates to estimate the frequency of hybridization (Table 3). We recovered an average of 9.67 double-drug-resistant tetraploids out of a calculated 6100 average viable cells per plate, or 0.158%. Flow cytometry of representative putative hybrids indicates DNA content consistent with tetraploidy when compared against known standards (FIG. 1A, FIG. 6). We note that induction of HO in diploids produces several possible genotypes by gene conversion, including MATa/MATa, MATα/MATα, and MATa/MATα diploids, but the HyPr method is expected to only select for those progeny that result from the mating of MATa/MATa diploids with MATα/MATα diploids that contain complementary marked plasmids. The HyPr method does not control which mating type is contributed by which parent.

TABLE 3
Recovery of double-drug-resistant colonies from
induced, co-cultured S. cerevisiae strains

| Replicate | YPD + hyg + nat | YPD¹ |
|---|---|---|
| A | 12 | 5200 |
| B | 7 | 6900 |
| C | 10 | 6200 |
| Mean ± SD | 9.67 ± 2.52 | 6100 ± 850 |

¹Total YPD colonies were calculated from actual colony counts using dilution factors.

Interspecies Tetraploids are Selectable from Co-Cultured, HO-Induced Diploids

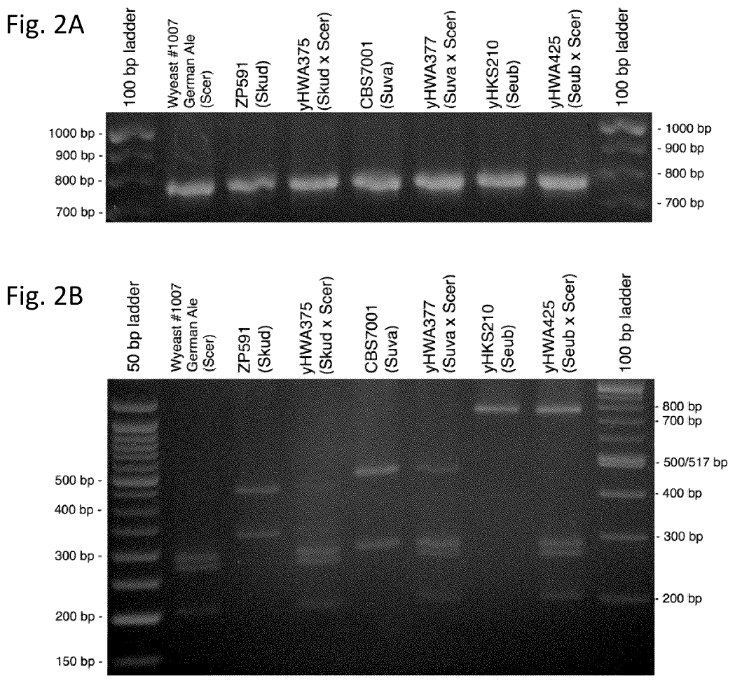

A German Ale strain of S. cerevisiae was transformed with pHCT2, while strains of S. kudriavzevii, S. uvarum, and S. eubayanus were transformed with pHMK34 (Table 1). HO expression was induced with doxycycline, and strains possessing complementary plasmids were co-cultured on YPD for up to 36 hours at room temperature. Patches were struck to YPD +hyg +nat, and resulting single double-drug-resistant colonies were grown in liquid YPD. Flow cytometry indicated a DNA content consistent with tetraploidy (FIGS. 1B-1D, FIG. 6), and PCR-RFLP analysis confirmed these strains were hybrids between S. cerevisiae and the intended Saccharomyces species (FIGS. 2A-2B).

### Plasmids are Rapidly Lost in Non-Selective Media

A saturated liquid non-selective culture of yHWA425, the synthetic S. cerevisiae x S. eubayanus hybrid (Table 1), was diluted 10⁻⁴, and 1 μL was plated onto each of three YPD plates. After incubation at room temperature for two days, these plates were replicated to YPD +hyg and YPD +nat plates. 54.9% of colonies lost pHMK34, while 63.8% of colonies lost pHCT2. Some colonies grew on YPD and were sensitive to both drugs (FIG. 7). These colonies were harvested from the YPD plate and were successfully struck for single colonies on YPD but not on YPD +hyg and YPD +nat (yHWA439), indicating that plasmid-free interspecies hybrids containing unmarked nuclear genomes can be readily obtained by this method.

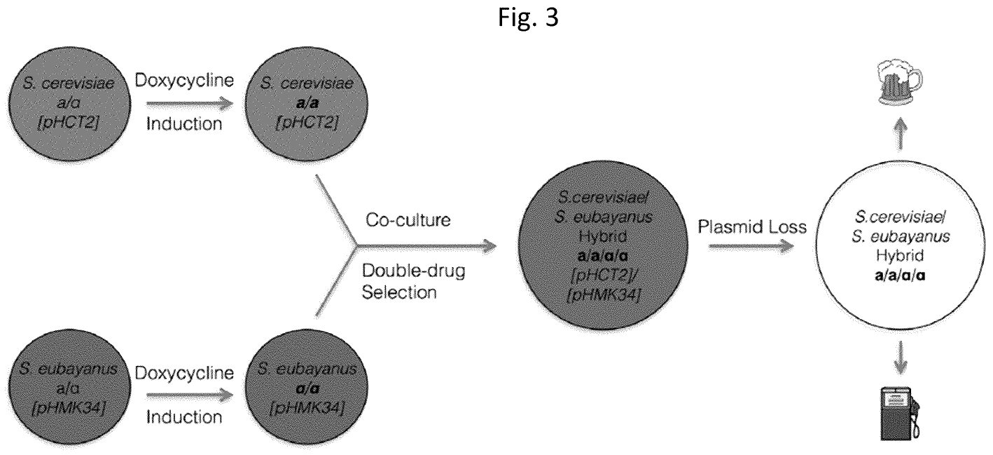

pHCT2 and pHMK34 function similarly to previously constructed vectors that drive HO expression with the galactose-inducible promoter of GAL1 (Jensen and Herskowitz, 1984) and are valuable tools for researchers working with stable haploid prototrophic strains of Saccharomyces. More interestingly, pHCT2 and pHMK34 can be used together in a method called HyPr, which produces allotetraploid and autotetraploid Saccharomyces yeasts (FIG. 3). The HyPr success rate is high (around 1 out of 1000 plated cells) relative to the corresponding rate of rare mating between two diploids in a population, which has been estimated to be between 1 out of 1 million to 100 million cells (Gunge and Nakatomi, 1972). Moreover, the complementary markers on the plasmids themselves also provide the means to easily select allotetraploids. The high success rate, the ease of use in prototrophs, the lack of permanent genomic modification, and the capability of plasmid loss together make HyPr the ideal method for production of new designer hybrid strains for industrial fermentations, such as beer, wine, cider, and biofuel production. To introduce the utility of this approach, we have created novel S. cerevisiae x S. eubayanus, S. cerevisiae x S. kudriavzevii, and S. cerevisiae x S. uvarum strains, which are designed as synthetic lager, Belgian, and cider strains, respectively. We also created novel allohexaploid strains of S. cerevisiae x S. eubayanus x S. kudriavzevii, as well as six-species hybrids of S. cerevisiae x S. paradoxus x S. arboricola x S. uvarum x S. kudriavzevii x S. mikatae inferred to have 12 sets of chromosomes.

HyPr may also provide an alternative method for optimization via hybridization of Saccharomyces chassis strains to be used in a variety of synthetic biology applications. Selection of an appropriate chassis strain prior to the installation of genetic and metabolic modifications is a sound strategy, but prior work has often focused on screening existing strain libraries to find a chassis strain that fit a set of criteria (Jin et al., 2013). The meiotic sterility of many industrial strains of interest complicates the production of new Saccharomyces chassis strains because many strains do not produce viable spores. HyPr can be used to create chassis strains de novo from two strains with desirable traits, producing a new chassis strain with both desired characteristics without sporulation. As with a previous case where HO was induced in a sterile strain whose genome was predominantly S. uvarum (Schwartz et al., 2012), HyPr is expected to even enable hybridization of strains whose defect is in sporulation or chromosome segregation, rather than mating per se. This new chassis strain could then undergo optimization via selective conditions, further enhancing desired phenotypes of the chassis strain through aneuploidy and other mutations or through modification using various genome-editing techniques (Alexander et al., 2014; DiCarlo et al., 2013; Ryan et al., 2014).

Chassis strains made by HyPr lack drug markers and auxotrophies, both of which are desirable qualities in beverage and biofuel strains. Note that, while integration of any portion of the plasmid into the Saccharomyces genome is highly unlikely, it is formally possible that fragments of the plasmid could have integrated and conferred no detectable phenotype. Depending on the desired applications and safety regulations, any hybrids made using HyPr could have their genomes sequenced to ensure that all plasmid DNA had been eliminated from the strain, or other routine molecular approaches, such as PCR or Southern blots, could be taken to verify the absence of specific parts of the plasmid that were of concern.

Extensive prior work has shown that S. cerevisiae autotetraploid strains are relatively unstable, rapidly losing chromosomes to form aneuploid strains (Storchova, 2014). This loss occurs rapidly with genome content reduction to near-diploid levels in 200 to 800 generations (Gerstein et al., 2006). We expect autotetraploids made via HyPr to behave in a similar manner. This could, in fact, be a desirable trait for many applications, as placing an unstable tetraploid strain in a selective condition will influence which components of the genome are retained or lost from which parent, allowing for more rapid adaptation to that condition (Selmecki et al., 2015). Allopolyploids between Saccharomyces species have been identified from many different sources, including the isolation of lager beer strains (Dunn and Sherlock, 2008; Nakao et al., 2009; Walther et al., 2014). The reduction of genomic components has been observed in many cases. For example, S. cerevisiae x S. kudriavzevii hybrids isolated from brewing environments have selectively lost components of the S. kudriavzevii genome (Peris et al., 2012c), while certain S. cerevisiae x S. eubayanus hybrids used in lager beer production have lost whole S. cerevisiae chromosomes (Walther et al., 2014). We expect that allotetraploids made by HyPr will also evolve aneuploidy given enough time, but again, many of these aneuploidies may be advantageous in the conditions where they are evolved. A recent evolution experiment using interspecies hybrids demonstrated that genomic stability was reached within 30-50 generations (Pérez-Través et al., 2014a). Even though allopolyploid strains are less stable than diploid Saccharomyces strains, we note that the allopolyploid strains that form the backbone of the brewing industry are sufficiently stable for routine application in large-scale fermentations.

Finally, interspecies hybrids are also useful to address a variety of basic research questions in genetics and evolutionary biology. For example, interspecies hybrids have been especially useful for examining the relative effects of cis and trans variation on gene expression (Bullard et al., 2010; Swain Lenz et al., 2014; Tirosh et al., 2009; Wittkopp et al., 2004). In Saccharomyces, these studies have traditionally mated haploids with integrated complementary markers to make F1 diploid hybrids. Our plasmid-based strategy may be preferable in strain backgrounds where gene targeting is inefficient or for high-throughput experiments. When diploidy is required, allotetraploid Saccharomyces that have not yet evolved aneuploidy or sterility can readily be sporulated readily to recover diploids (Greig et al., 2002; Gunge, 1966). In conclusion, this straightforward, robust approach allows the efficient construction of designer hybrids of Saccharomyces allotetraploids and autotetraploids, as well as strains with higher order ploidy, for numerous basic and applied uses.

### REFERENCES

Alexander, W. G., Doering, D. T., Hittinger, C. T., 2014. High-efficiency genome editing and allele replacement in prototrophic and wild strains of Saccharomyces. Genetics 198, 859-866. doi:10.1534/genetics.114.170118
Almeida, P., Gonçalves, C., Teixeira, S., Libkind, D., Bontrager, M., Masneuf-Pomarède, I., Albertin, W., Durrens, P., Sherman, D. J., Marullo, P., Hittinger, C. T., Gonçalves, P., Sampaio, J. P., 2014. A Gondwanan imprint on global diversity and domestication of wine and cider yeast Saccharomyces uvarum. Nat. Commun. 5, 4044. doi:10.1038/ncomms5044
Baker, E., Wang, B., Bellora, N., Peris, D., Hulfachor, A. B., Koshalek, J. A., Adams, M., Libkind, D., Hittinger, C. T., 2015. The genome sequence of Saccharomyces eubayanus and the domestication of lager-brewing yeasts. Mol. Biol. Evol. msv168-. doi:10.1093/molbev/msv168
Bardwell, L., 2004. A walk-through of the yeast mating pheromone response pathway. Peptides 25, 1465-1476. doi:10.1016/j.peptides.2003.10.022
Bing, J., Han, P.-J., Liu, W.-Q., Wang, Q.-M., Bai, F.-Y., 2014. Evidence for a Far East Asian origin of lager beer yeast. Curr. Biol. 24, R380-1. doi:10.1016/j.cub.2014.04.031
Brem, R. B., Yvert, G., Clinton, R., Kruglyak, L., 2002. Genetic dissection of transcriptional regulation in budding yeast. Science 296, 752-755. doi:10.1126/science.1069516
Bullard, J. H., Mostovoy, Y., Dudoit, S., Brem, R. B., 2010. Polygenic and directional regulatory evolution across pathways in Saccharomyces. Proc. Natl. Acad. Sci. U.S.A 107, 5058-63. doi:10.1073/pnas.0912959107
Casselton, L. A., Olesnicky, N. S., 1998. Molecular genetics of mating recognition in basidiomycete fungi. Microbiol. Mol. Biol. Rev. 62, 55-70.
Corran, H. S., 1975. A history of brewing. David & Charles.
DiCarlo, J. E., Norville, J. E., Mali, P., Rios, X., Aach, J., Church, G. M., 2013. Genome engineering in Saccharomyces cerevisiae using CRISPR-Cas systems. Nucleic Acids Res. 41, 4336-43. doi:10.1093/nar/gktl35
Dunn, B., Sherlock, G., 2008. Reconstruction of the genome origins and evolution of the hybrid lager yeast Saccharomyces pastorianus. Genome Res. 18, 1610-1623. doi:10.1101/gr.076075.108
Fabiani, L., Irene, C., Aragona, M., Newlon, C. S., 2001. A DNA replication origin and a replication fork barrier used in vivo in the circular plasmid pKD1. Mol. Genet. Genomics 266, 326-35.
Fortuna, M., Joao Sousa, M., Corte-Real, M., Leao, C., 1997. UNIT 11.13 Cell cycle analysis of yeasts, in: Current Protocols in Cytometry.
Frank-Vaillant, M., Marcand, S., 2001. NHEJ regulation by mating type is exercised through a novel protein, Lif2p, essential to the ligase IV pathway. Genes Dev. 15, 3005-12. doi:10.1101/gad.206801
Garí, E., Piedrafita, L., Aldea, M., Herrero, E., 1997. A set of vectors with a tetracycline-regulatable promoter system for modulated gene expression in Saccharomyces cerevisiae. Yeast 13, 837-48. doi:10.1002/(SICI)1097-0061(199707)13:9<837::AID-YEA145>3.0.CO;2-T
Gerstein, A. C., Chun, H.-J. E., Grant, A., Otto, S. P., 2006. Genomic convergence toward diploidy in Saccharomyces cerevisiae. PLoS Genet. 2, e145. doi:10.1371/journal.pgen.0020145
Gietz, R. D., Woods, R. A., 2002. Transformation of yeast by lithium acetate/single-stranded carrier DNA/polyethylene glycol method. Methods Enzymol. 350, 87-96.
Glass, N. L., Grotelueschen, J., Metzenberg, R. L., 1990. Neurospora crassa A mating-type region. Proc. Natl. Acad. Sci. U.S.A 87, 4912-4916.
Glass, N. L., Jacobson, D. J., Shiu, P. K. T., 2000. The genetics of hyphal fusion and vegetative incompatibility in filamentous ascomycete fungi. Annu. Rev. Genet. 34, 165-186.
Goldstein, A. L., McCusker, J. H., 1999. Three new dominant drug resistance cassettes for gene disruption in Saccharomyces cerevisiae. Yeast 15, 1541-53. doi:10.1002/(SICI)1097-0061(199910)15:14<1541::AID-YEA476>3.0.CO;2-K
González, S. S., Barrio, E., Querol, A., 2008. Molecular characterization of new natural hybrids of Saccharomyces cerevisiae and S. kudriavzevii in brewing. Appl. Environ. Microbiol. 74, 2314-2320. doi:10.1128/AEM.01867-07
Greig, D., Borts, R. H., Louis, E. J., Travisano, M., 2002. Epistasis and hybrid sterility in Saccharomyces. Proc. Biol. Sci. 269, 1167-71. doi:10.1098/rspb.2002.1989
Gunge, N., 1966. Breeding of bakers' yeast-Determination of the ploidy and an attempt to improve practical properties. Japanese J. Genet. 41, 203-214. doi:10.1266/jjg.41.203
Gunge, N., Nakatomi, Y., 1972. Genetic mechanisms of rare matings of the yeast Saccharomyces cerevisiae heterozygous for mating type. Genetics 70, 41-58.
Haber, J. E., 2012. Mating-type genes and MAT switching in Saccharomyces cerevisiae. Genetics 191, 33-64. doi:10.1534/genetics.111.134577
Hebly, M., Brickwedde, A., Bolat, I., Driessen, M. R. M., de Hulster, E. A. F., van den Broek, M., Pronk, J. T., Geertman, J.-M., Daran, J.-M., Daran-Lapujade, P., 2015. S. cerevisiae x S. eubayanus interspecific hybrid, the best of both worlds and beyond. FEMS Yeast Res. 15. doi:10.1093/femsyr/fov005
Herskowitz, I., Jensen, R. E., 1991. Putting the HO gene to work: practical uses for mating-type switching. Methods Enzymol. 194, 132-46.
Hittinger, C. T., 2013. Saccharomyces diversity and evolution: a budding model genus. Trends Genet. 29, 309-17. doi:10.1016/j.tig.2013.01.002
Hittinger, C. T., Gonçalves, P., Sampaio, J. P., Dover, J., Johnston, M., Rokas, A., 2010. Remarkably ancient balanced polymorphisms in a multi-locus gene network. Nature 464, 54-8. doi:10.1038/nature08791
Husnik, J. I., Volschenk, H., Bauer, J., Colavizza, D., Luo, Z., van Vuuren, H. J. J., 2006. Metabolic engineering of malolactic wine yeast. Metab. Eng. 8, 315-23. doi:10.1016/j.ymben.2006.02.003
Jensen, R. E., Herskowitz, I., 1984. Directionality and regulation of cassette substitution in yeast. Cold Spring Harb. Symp. Quant. Biol. 49, 97-104. doi:10.1101/SQB.1984.049.01.013
Jin, M., Sarks, C., Gunawan, C., Bice, B. D., Simonett, S. P., Avanasi Narasimhan, R., Willis, L. B., Dale, B. E., Balan, V., Sato, T. K., 2013. Phenotypic selection of a wild Saccharomyces cerevisiae strain for simultaneous saccharification and co-fermentation of AFEX™ pretreated corn stover. Biotechnol. Biofuels 6, 108. doi:10.1186/1754-6834-6-108
John, B., 1990. Meiosis, Developmental and Cell Biology Series.
Kohl, K. P., Sekelsky, J., 2013. Meiotic and mitotic recombination in meiosis. Genetics 194, 327-34. doi:10.1534/genetics.113.150581
Krogerus, K., Magalhães, F., Vidgren, V., Gibson, B., 2015. New lager yeast strains generated by interspecific hybridization. J. Ind. Microbiol. Biotechnol. 42, 769-78. doi:10.1007/s10295-015-1597-6
Kronstad, J. W., Staben, C., 1997. Mating type in filamentous fungi. Annu. Rev. Genet. 31, 245-76. doi:10.1146/annurev.genet.31.1.245
Le Jeune, C., Lollier, M., Demuyter, C., Erny, C., Legras, J.-L., Aigle, M., Masneuf-Pomarède, I., 2007. Characterization of natural hybrids of Saccharomyces cerevisiae and Saccharomyces bayanus var. uvarum. FEMS Yeast Res. 7, 540-9. doi:10.1111/j.1567-1364.2007.00207.x
Leducq, J.-B., Charron, G., Samani, P., Dubé, A. K., Sylvester, K., James, B., Almeida, P., Sampaio, J. P., Hittinger, C. T., Bell, G., Landry, C. R., 2014. Local climatic adaptation in a widespread microorganism. Proc. Biol. Sci. 281, 20132472. doi:10.1098/rspb.2013.2472
Libkind, D., Hittinger, C. T., Valério, E., Gonçalves, C., Dover, J., Johnston, M., Gonçalves, P., Sampaio, J. P., 2011. Microbe domestication and the identification of the wild genetic stock of lager-brewing yeast. Proc. Natl. Acad. Sci. U.S.A 108, 14539-44. doi:10.1073/pnas.1105430108
Liti, G., Carter, D. M., Moses, A. M., Warringer, J., Parts, L., James, S. a, Davey, R. P., Roberts, I. N., Burt, A., Koufopanou, V., Tsai, I. J., Bergman, C. M., Bensasson, D., O'Kelly, M. J. T., van Oudenaarden, A., Barton, D. B. H., Bailes, E., Nguyen, A. N., Jones, M., Quail, M. a, Goodhead, I., Sims, S., Smith, F., Blomberg, A., Durbin, R., Louis, E. J., 2009. Population genomics of domestic and wild yeasts. Nature 458, 337-41. doi:10.1038/nature07743
Liti, G., Peruffo, A., James, S. a, Roberts, I. N., Louis, E. J., 2005. Inferences of evolutionary relationships from a population survey of LTR-retrotransposons and telomeric-associated sequences in the Saccharomyces sensu stricto complex. Yeast 22, 177-92. doi:10.1002/yea.1200
Longtine, M. S., Mckenzie III, A., Demarini, D. J., Shah, N. G., Wach, A., Brachat, A., Philippsen, P., Pringle, J. R., 1998. Additional modules for versatile and economical PCR-based gene deletion and modification in Saccharomyces cerevisiae. Yeast 14, 953-961.
Maclean, C. J., Greig, D., 2008. Prezygotic reproductive isolation between Saccharomyces cerevisiae and Saccharomyces paradoxus. BMC Evol. Biol. 8, 1. doi:10.1186/1471-2148-8-1
Masneuf, I., Hansen, J., Groth, C., Piskur, J., Dubourdieu, D., 1998. New hybrids between Saccharomyces sensu stricto yeast species found among wine and cider production strains. Appl. Envir. Microbiol. 64, 3887-3892.
Mortimer, R. K., 2000. Evolution and variation of the yeast (Saccharomyces) genome. Genome Res. 10, 403-409. doi:10.1101/gr.10.4.403
Mortimer, R. K., Johnston, J. R., 1986. Genealogy of principal strains of the yeast genetic stock center. Genetics 113, 35-43.
Nakao, Y., Kanamori, T., Itoh, T., Kodama, Y., Rainieri, S., Nakamura, N., Shimonaga, T., Hattori, M., Ashikari, T., 2009. Genome sequence of the lager brewing yeast, an interspecies hybrid. DNA Res. 16, 115-29. doi:10.1093/dnares/dsp003
Pérez-Través, L., Lopes, C. A., Barrio, E., Querol, A., 2014a. Stabilization process in Saccharomyces intra and interspecific hybrids in fermentative conditions. Int. Microbiol. 17, 213-24. doi:10.2436/20.1501.01.224
Pérez-Través, L., Lopes, C. A., Barrio, E., Querol, A., 2012. Evaluation of different genetic procedures for the generation of artificial hybrids in Saccharomyces genus for winemaking. Int. J. Food Microbiol. 156, 102-11. doi:10.1016/j.ijfoodmicro.2012.03.008
Pérez-Través, L., Lopes, C. A., Querol, A., Barrio, E., 2014b. On the complexity of the Saccharomyces bayanus taxon: hybridization and potential hybrid speciation. PLoS One 9, e93729. doi:10.1371/journal.pone.0093729
Peris, D., Belloch, C., Lopandić, K., Álvarez-Pérez, J. M., Querol, A., Barrio, E., 2012a. The molecular characterization of new types of Saccharomyces cerevisiae x S. kudriavzevii hybrid yeasts unveils a high genetic diversity. Yeast 29, 81-91. doi:10.1002/yea.2891
Peris, D., Lopes, C. A., Arias, A., Barrio, E., 2012b. Reconstruction of the evolutionary history of Saccharomyces cerevisiae x S. kudriavzevii hybrids based on multilocus sequence analysis. PLoS One 7, e45527. doi:10.1371/journal.pone.0045527
Peris, D., Lopes, C. A., Belloch, C., Querol, A., Barrio, E., 2012c. Comparative genomics among Saccharomyces cerevisiae x Saccharomyces kudriavzevii natural hybrid strains isolated from wine and beer reveals different origins. BMC Genomics. doi:10.1186/1471-2164-13-407
Peris, D., Sylvester, K., Libkind, D., Gonçalves, P., Sampaio, J. P., Alexander, W. G., Hittinger, C. T., 2014. Population structure and reticulate evolution of Saccharomyces eubayanus and its lager-brewing hybrids. Mol. Ecol. 23, 2031-45. doi:10.1111/mec.12702
Piatkowska, E. M., Naseeb, S., Knight, D., Delneri, D., 2013. Chimeric protein complexes in hybrid species generate novel phenotypes. PLoS Genet. 9, e1003836. doi:10.1371/journal.pgen.1003836
Piotrowski, J. S., Nagarajan, S., Kroll, E., Stanbery, A., Chiotti, K. E., Kruckeberg, A. L., Dunn, B., Sherlock, G., Rosenzweig, F., 2012. Different selective pressures lead to different genomic outcomes as newly-formed hybrid yeasts evolve. BMC Evol. Biol. 12, 46. doi:10.1186/1471-2148-12-46
Russell, D. W., Jensen, R., Zoller, M. J., Burke, J., Errede, B., Smith, M., Herskowitz, I., 1986. Structure of the Saccharomyces cerevisiae HO gene and analysis of its upstream regulatory region. Mol. Cell. Biol. 6, 4281-94.
Ryan, O. W., Skerker, J. M., Maurer, M. J., Li, X., Tsai, J. C., Poddar, S., Lee, M. E., DeLoache, W., Dueber, J. E., Arkin, A. P., Cate, J. H. D., 2014. Selection of chromosomal DNA libraries using a multiplex CRISPR system. Elife e03703. doi:10.7554/eLife.03703
Sabina, J., Johnston, M., 2009. Asymmetric signal transduction through paralogs that comprise a genetic switch for sugar sensing in Saccharomyces cerevisiae. J. Biol. Chem. 284, 29635-29643. doi:10.1074/jbc.M109.032102
Sampaio, J. P., Gonçalves, P., 2008. Natural populations of Saccharomyces kudriavzevii in Portugal are associated with oak bark and are sympatric with S. cerevisiae and S. paradoxus. Appl. Environ. Microbiol. 74, 2144-2152. doi:10.1128/AEM.02396-07
Scannell, D. R., Zill, O. A., Rokas, A., Payen, C., Dunham, M. J., Eisen, M. B., Rine, J., Johnston, M., Hittinger, C. T., 2011. The awesome power of yeast evolutionary genetics: new genome sequences and strain resources for the Saccharomyces sensu stricto genus. G3 1, 11-25. doi:10.1534/g3.111.000273
Schwartz, K., Wenger, J. W., Dunn, B., Sherlock, G., 2012. APJ1 and GRE3 homologs work in concert to allow growth in xylose in a natural Saccharomyces sensu stricto hybrid yeast. Genetics 191, 621-32. doi:10.1534/genetics.112.140053
Selmecki, A. M., Maruvka, Y. E., Richmond, P. A., Guillet, M., Shoresh, N., Sorenson, A. L., De, S., Kishony, R., Michor, F., Dowell, R., Pellman, D., 2015. Polyploidy can drive rapid adaptation in yeast. Nature 519, 349-52. doi:10.1038/nature14187
Staben, C., Yanofsky, C., 1990. Neurospora crassa a mating-type region. Proc. Natl. Acad. Sci. U.S.A 87, 4917-21.
Storchova, Z., 2014. Ploidy changes and genome stability in yeast. Yeast 31, 421-30. doi:10.1002/yea.3037
Swain Lenz, D., Riles, L., Fay, J. C., 2014. Heterochronic meiotic misexpression in an interspecific yeast hybrid. Mol. Biol. Evol. 31, 1333-1342. doi:10.1093/molbev/msu098
Tirosh, I., Reikhav, S., Levy, A. A., Barkai, N., 2009. A yeast hybrid provides insight into the evolution of gene expression regulation. Science 324, 659-62. doi:10.1126/science.1169766
Urlinger, S., Baron, U., Thellmann, M., Hasan, M. T., Bujard, H., Hillen, W., 2000. Exploring the sequence space for tetracycline-dependent transcriptional activators: novel mutations yield expanded range and sensitivity. Proc. Natl. Acad. Sci. U.S.A 97, 7963-8. doi:10.1073/pnas.130192197
Van Heeckeren, W. J., Dorris, D. R., Struhl, K., 1998. The mating-type proteins of fission yeast induce meiosis by directly activating mei3 transcription. Mol. Cell. Biol. 18, 7317-26.
Walker, M. E., Gardner, J. M., Vystavelova, A., McBryde, C., Lopes, M. D. B., Jiranek, V., 2003. Application of the reuseable, KanMX selectable marker to industrial yeast: Construction and evaluation of heterothallic wine strains of Saccharomyces cerevisiae, possessing minimal foreign DNA sequences. FEMS Yeast Res. 4, 339-347. doi:10.1016/S1567-1356(03)00161-2
Walther, A., Hesselbart, A., Wendland, J., 2014. Genome sequence of Saccharomyces carlsbergensis, the world's first pure culture lager yeast. G3 4, 783-793. doi:10.1534/g3.113.010090
Wang, Q.-M., Liu, W.-Q., Liti, G., Wang, S.-A., Bai, F.-Y., 2012. Surprisingly diverged populations of Saccharomyces cerevisiae in natural environments remote from human activity. Mol. Ecol. 21, 5404-17. doi:10.1111/j.1365-294X.2012.05732.x
Wittkopp, P. J., Haerum, B. K., Clark, A. G., 2004. Evolutionary changes in cis and trans gene regulation. Nature 430, 85-8. doi:10.1038/nature02698
Wohlbach, D. J., Rovinskiy, N., Lewis, J. A., Sardi, M., Schackwitz, W. S., Martin, J. A., Deshpande, S., Daum, C. G., Lipzen, A., Sato, T. K., Gasch, A. P., 2014. Comparative genomics of Saccharomyces cerevisiae natural isolates for bioenergy production. Genome Biol. Evol. 6, 2557-66.
Zerbino, D. R., Birney, E., 2008. Velvet: algorithms for de novo short read assembly using de Bruijn graphs. Genome Res. 18, 821-9. doi:10.1101/gr.074492.107

### Example 2—Generation of Higher Order Polyploid Synthetic Yeast Cells

Hybrid strain yHWA425 (S. cerevisiae x S. eubayanus) was passaged as described above such that it lost pHMK34 but retained pHCT2 (i.e. screening for nourseothricin-resistant but hygromycin-sensitive colonies by replica-plating). Strain yHWA425 was then crossed to strain yHWA354 (S. kudriavzevii), and triple hybrids resistant to both nourseothricin and hygromycin were selected using the protocols described above to generate allohexaploid strains yHWA451 and yHWA452 (S. cerevisiae x S. eubayanus x S. kudriavzevii). See Table 1. These triple hybrids were then passaged as described above such that both plasmids were lost, yielding yHWA455 and yHWA456 (S. cerevisiae x S. eubayanus x S. kudriavzevii). See Table 1. PCR and Restriction Fragment Long Polymorphism (RFLP) were used to confirm the presence of three different genomes (S. cerevisiae x S. kudriavzevii x S. eubayanus) in a synthetic higher ploidy hybrid (likely an allohexaploid) as shown in FIG. 8. The BRE5 PCR product was digested with HaeIII producing a pattern for each species contributing to this gene/genome, as described in FIGS. 2A-2B above.

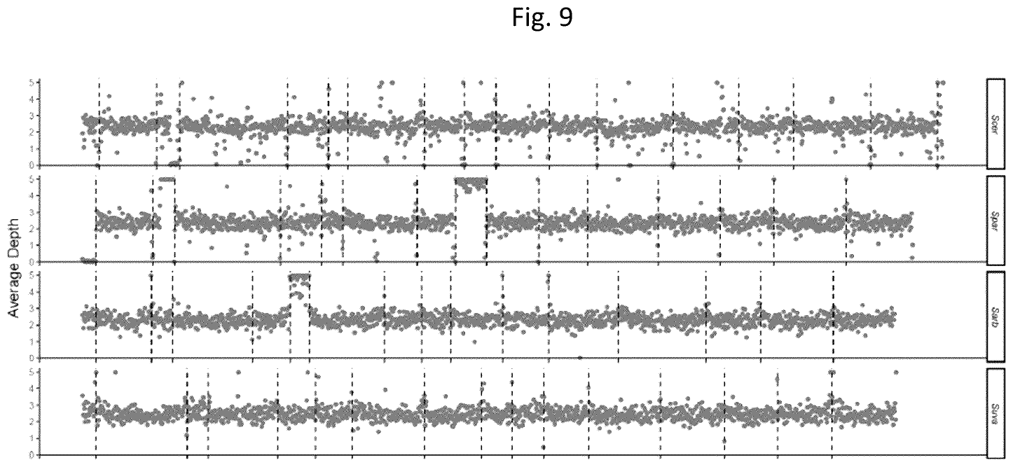

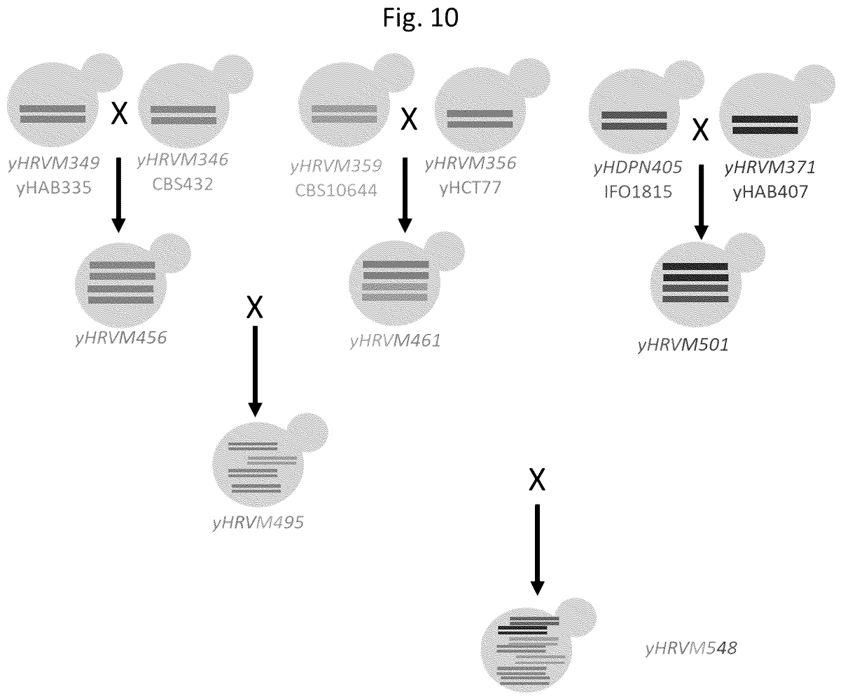

In addition to the hybrids described above, we were able to make higher order hybrids and have demonstrated both four and six species hybrids. These higher order hybrids were generated by taking two species hybrids such as those made above and further combining them. We generated several two-species hybrids, including S. cerevisiae x S. paradoxus, S. arboricola x S. uvarum and S. mikatae x S. kudriavzevii. Two of these two-species hybrids were then crossed and a four-species hybrid was selected. The four-species hybrid was confirmed by Illumina sequencing and analysis (as previously described in Peris et al. 2017 Biotechnol Biofuels 10:78) by comparison to the parents as shown in FIG. 9. As shown in FIG. 10, the four-species hybrid is a hybrid containing genetic material from S. cerevisiae, S. paradoxus, S. arboricola, S. uvarum. This four-species hybrid (S. cerevisiae x S. paradoxus x S. arboricola x S. uvarum) was then crossed with the two-species hybrid (S. mikatae x S. kudriavzevii) and a six-species hybrid was selected for and screened using Restriction Fragment Length Polymorphism analysis as shown in FIG. 11B to demonstrate that the hybrid was a 6-species hybrid (S. cerevisiae x S. paradoxus x S. arboricola x S. uvarum x S. mikatae x S. kudriavzevii). A photograph showing the diverse morphology of the 6 species hybrid is provided as FIG. 11A. This suggest chromosome instability, but work is continuing to demonstrate the variation within and ability to select for traits from these higher order hybrids. Thus, higher order hybrids can be made using the techniques described herein.

We claim:

1. A synthetic yeast cell comprising whole genomes from at least 3 different yeast species.

2. The synthetic yeast cell of claim 1, wherein the synthetic yeast cell comprises 3 to 16 sets of yeast chromosomes.

3. The synthetic yeast cell of claim 1, wherein the synthetic yeast cell comprises 6 to 12 sets of yeast chromosomes.

4. The synthetic yeast cell of claim 1, wherein the synthetic yeast cell comprises chromosomes or chromosomal segments from 4-6 different yeast species.

5. The synthetic yeast cell of claim 1, wherein the synthetic yeast cell comprises chromosomes or chromosomal segments from an ascomycete.

6. The synthetic yeast cell of claim 1, wherein synthetic yeast cell comprises chromosomes or chromosomal segments from at least one Saccharomyces species.

7. The synthetic yeast cell of claim 6, wherein the at least one Saccharomyces species is selected from Saccharomyces cerevisiae, Saccharomyces paradoxus, Saccharomyces mikatae, Saccharomyces jurei, Saccharomyces arboricola, Saccharomyces kudriavzevii, Saccharomyces uvarum, and Saccharomyces eubayanus.

8. The synthetic yeast cell of claim 1, wherein the synthetic yeast cell comprises chromosomes or chromosomal segments from a yeast strain selected from RM11-1a, Ethanol Red, NRRL YB-210, Wyeast #1007 German Ale, ZP 591, CBS 7001, yHKS210, White Labs WLP830 German Lager, yHWA338, yHWA340, yHWA341, yHWA348, yHWA350, yHWA352, yHWA354, yHWA358, yHWA375, yHWA377, yHWA425, yHWA439, yHWA451, yHWA452, yHWA453, yHWA454, yHWA455, yHWA456, yHWA457, yHWA458, yHWA459, yHWA460, yHRVM349, yHRVM346, yHRVM359, yHRVM356, yHDPN405, yHRVM371, yHRVM456, yHRVM461, yHRVM 501, yHRVM495, and yHRVM548.

9. A method comprising using the synthetic yeast cell of claim 1 in a fermentation process.

10. The method of claim 9, wherein the fermentation process is selected from the group consisting of beer making, wine making, biochemical production, bioremediation, and biofuel production.
